(19) United States
(12) Patent Application Publication
(10) Pub. No.: US 2006/0148712 A1
(43) Pub. Date: Jul. 6, 2006

(54) Monocyte chemoattractant activity of galectin-3

(76) Inventors: Fu-Tong Liu, Davis, CA (US); Hideki Sano, Chiba (JP); Daniel K. Hsu, Davis, CA (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 11/288,966

(22) Filed: Nov. 28, 2005

Related U.S. Application Data
(63) Continuation-in-part of application No. 09/805,449, filed on Mar. 13, 2001.
(60) Provisional application No. 60/188,795, filed on Mar. 13, 2000.

(51) Int. Cl. A61K 38/10

(52) U.S. Cl. 514/13

(57) ABSTRACT

Inhibitors of galectin-3 expression or activity, for administering to a subject in an amount sufficient to reduce or decrease onset, progression, severity, frequency, duration or probability of one or more symptoms associated with asthma, among other respiratory airway and respiratory mucosal disorders.

### RELATED APPLICATIONS

This application is a continuation-in-part and claims priority to application Ser. No. 09/805,449, filed Mar. 13, 2001, and application Ser. No. 60/188,795, filed Mar. 13, 2000, each of which are expressly incorporated herein by reference.

### GOVERNMENT RESEARCH

This invention was made with Government support under Grant No. A139620, awarded by the NIH. The Government may have certain rights in the invention.

### FIELD OF THE INVENTION

The present invention relates to methods for modulating migration of cells, especially monocytes, neutrophils and macrophages, using galectin-3, galectin-3 binding polypeptides, galectin-3 receptor binding polypeptides or galectin-3 mimetics. The invention also relates to screening methods for identifying agents that modulate galectin-3-mediated cell migration.

### BACKGROUND OF THE INVENTION

Lectins are proteins that bind to specific carbohydrate structures and can thus recognize particular glycoconjugates. Galectins are a family of over 10 structurally related lectins that bind beta-galactosides.

Galectin-3 is a 26 kDa beta-galactoside-binding protein belonging to the galectin family. This protein is composed of a carboxyl-terminal carbohydrate-recognition domain (CRD) and amino-terminal tandem repeats. Galectin-3 is found in epithelia of many organs, as well as in various inflammatory cells, including macrophages, dendritic cells and Kupffer cells. The expression of galectin-3 is upregulated during inflammation, cell proliferation, cell differentiation, and through transactivation by viral proteins. Its expression is also affected by neoplastic transformation—upregulated in certain types of lymphomas and thyroid carcinoma; downregulated in other types of malignancies, such as colon, breast, ovarian and uterine carcinomas. Recently, it has been reported that the expression of this lectin has a strong correlation with the grade and malignant potential of primary brain tumors. Increased galectin-3 expression has also been noted in human atherosclerotic lesions. These findings suggest that galectin-3 may mediate both physiological and pathological responses.

Galectin-3 has been shown to function through both intracellular and extracellular actions. Related to its intracellular functions, galectin-3 has been identified as a component of hnRNP, a factor in pre-mRNA splicing. Intracellular galectin-3 has also been found to exert cell cycle control and prevent T cell apoptosis, the latter probably mediated through interaction with the Bcl-2 family members. Extracellular forms of galectin-3 secreted from monocytes/macrophages and epithelial cells, function in the activation of various types of cells, including monocytes/macrophages, mast cells, neutrophils, and lymphocytes. Galectin-3 has also been shown to mediate cell-cell and cell-extracellular matrix interactions.

Galectin-9, another member of the galectin family with two CRDs, is a selective chemoattractant for eosinophils. The activity requires both CRDs, suggesting that cross-linking of cell surface molecules is involved in the chemoattraction. Galectin-3 is known to form dimers through the amino-terminal non-lectin domain and thus has the potential to cross-link appropriate cell surface glycoproteins.

Extracellularly, galectin-3 is known to bind to the cell surfaces of monocytes/macrophages. High levels of galectin-3 expression are seen in human and rat lungs, where macrophages are one of the dominant cell types. Moreover, the recruitment of macrophages during peritonitis has been found to be attenuated in galectin-3-deficient mouse.

### SUMMARY OF THE INVENTION

The present invention provides a method for modulating migration of a cell that expresses a galectin-3 receptor comprising contacting the cell with a migration-modulating amount of galectin-3, galectin-3 binding polypeptide, or galectin-3 receptor binding polypeptide.

Also provided is a method for modulating monocyte, neutrophil or macrophage migration comprising contacting a monocyte or macrophage with a migration-modulating amount of galectin-3, galectin-3 binding polypeptide, or galectin-3 receptor binding polypeptide.

According to these methods, the migration may be stimulated or inhibited. Further, the galectin-3 may comprise an N-terminal or C-terminal subsequence of galectin-3, while the galectin-3 binding polypeptide may be a galectin-3 antibody or binding fragment thereof. Preferably, migration is modulated in an animal.

The present invention also provides methods for increasing migration of monocytes, neutrophils or macrophages to an inflammatory, infection or tumor site comprising contacting the inflammatory, infection or tumor site, respectively, with a migration-increasing amount of galectin-3, galectin-3 binding polypeptide, or galectin-3 receptor binding polypeptide.

In one embodiment, the invention provides a method for identifying an agent that modulates galectin-3 mediated cell migration comprising: contacting galectin-3 with a test agent; and detecting galectin-3 mediated cell migration, wherein an alteration of galectin-3 meditated cell migration in the presence of the test agent identifies an agent that modulates galectin-3 mediated cell migration. The agent may increase or decrease galectin-3 mediated cell migration, and may be, for example, a small molecule. Contacting according to this method may be in vitro, in cells or in vivo.

Also provided by the invention is an antibody that specifically binds galectin-3. Compositions comprising galectin-3 or a functional subsequence thereof and a pharmaceutically acceptable carrier, excipient or diluent or a drug are encompassed by the invention. The drug can, for example, be an anti-tumor, antiviral, antibacterial, anti-mycobacterial, anti-fungal, anti-cell proliferative or apoptotic agent.

Also included is a composition comprising galectin-3 or a functional subsequence thereof and an article of manufacture. The article of manufacture can be a dressing, such as a bandage, a suture, a sponge, or a surgical dressing.

The present invention also includes a microfabricated device containing galectin-3 or a functional subsequence thereof in a pharmaceutically acceptable carrier, said device capable of controlled delivery of the galectin-3 or the functional subsequence. According to this embodiment of the invention, the device can be implanted in the body of a subject at site of infection, in close proximity to or within a solid tumor, or at a site of a lesion.

### BRIEF DESCRIPTION OF THE DRAWINGS

FIG. 1 shows the effect of galectin-3 on human peripheral blood monocyte migration in vitro. Various concentrations of galectin-3 [and MCP-1 (100 ng/ml) as a positive control] were applied to the lower chambers of a micro Boyden chamber, purified monocytes were applied to the upper chambers, and the migration assay was performed. Methods. Data are the mean±SD of 4 individual experiments.

FIG. 2 illustrates the effect of anti-galectin-3 mAb on monocyte migration. After treatment with control (●) or anti-galectin-3 (∘) mAb, purified monocytes were added to the upper chambers and the migration assay was performed as described in Materials and Methods. Data are the mean±SD of 3 individual experiments.

FIG. 3 is a line graph depicting chemotaxis versus chemokinesis in galectin-3-activated monocytes. The data from the checkerboard experiment in Table 1 below has been represented graphically in this figure. Closed circles (●) represent the monocyte migration when galectin-3 was added only to the lower chambers. Open squares (□) show monocyte migration when equal concentrations of galectin-3 were added to both chambers.

FIG. 4A-4B show the effect of sugars on galectin-3-induced monocyte migration. Various concentrations of galectin-3 were mixed with 0 mM (●), 5 mM (▪), or 10 mM (▴) lactose (panel A) or sucrose (panel B) and placed in the lower chambers. Purified monocytes were added to the upper chambers and a standard migration assay was then performed. Data are the mean±SD of 4 individual experiments.

FIG. 5 is a line graph of the effect of a C-terminal domain fragment of galectin-3 (galectin-3C) on galectin-3-induced monocyte migration. After monocytes were incubated with the indicated concentrations of galectin-3C, the cells were added to the upper chambers and a standard migration assay was performed. Data are the mean±SD of 4 individual experiments.

FIG. 6A-6B are a pair of graphs comparing the effect of PTX on monocyte migration. After monocytes were treated with PTX, the cells were added to the upper chambers and the migration towards galectin-3 (panel A) or MCP-1 (panel B) was performed as described in Materials and Methods. Data are the mean±SD of 4 individual experiments.

FIG. 7 illustrates the effect of galectin-3 and MCP-1 on Ca²⁺ mobilization in monocytes. Traces represent the average mobilized intracellular concentrations of Ca²⁺ in the examined monocytes. The final concentrations of galectin-3 and MCP-1 in the cell suspensions were 1 μM and 100 ng/ml, respectively. Panels A and B: Effect of galectin-3 (A) and MCP-1 (B) on Ca²⁺ influx in monocytes, respectively. These reagents were added to the cell suspensions at 2 min after the initiation of the measurement. Panels C and D: Effect of two different sugars on galectin-3-induced Ca²⁺ influx in monocytes. After 5 mM lactose (C) or sucrose (D) was mixed with the cell suspension, galectin-3 and MCP-1 were added as the first and the second stimulants at 2 and 6 min after the start of the measurement. Panels E and F: Effect of PTX on galectin-3-induced Ca²⁺ influx in monocytes. Monocytes were incubated in the presence or absence of 1 μg/ml of PTX (together with Indo-1 AM) for 45 min prior to the assay. MCP-1 and galectin-3 were sequentially added to the monocyte suspensions, in the presence of the same concentration of PTX. Each figure shows representative data from 3 individual experiments using different donors.

FIG. 8 shows the effect of chemokines on galectin-3-induced Ca²⁺ mobilization in monocytes. Traces represent the average intracellular concentrations of Ca²⁺ in the examined monocytes. Monocytes were stimulated first with galectin-3 and then with MCP-1 (A), MIP-1α (C), or SDF-1α (E), or first with MCP-1 (B), MIP-1α (D), or SDF-1α (F) and then with galectin-3. The final concentrations of galectin-3 and each chemokine in the cell suspensions were 1 μM and 100 ng/ml, respectively. The first and the second stimulants were added to the cell suspension at 2 and 6 min after the start of the measurement. Each figure shows representative data from 3 individual experiments using different donors.

FIG. 9 is a bar graph illustrating the effect of galectin-3 and MCP-1 on the migration of cultured human peripheral blood macrophages in vitro. The assays were performed as described in FIG. 1. Data are the mean±SD of 3 individual experiments.

FIG. 10 depicts the effect of galectin-3 and MCP-1 on the migration of human alveolar macrophages in vitro. Alveolar macrophages obtained from bronchoalveolar lavage (BAL) fluid were used in a standard migration assay. The results from 2 separate experiments are shown.

FIG. 11 shows the effect of galectin-3 on monocyte/macrophage recruitment in mouse air pouches. One μM galectin-3 (●) (n=4), vehicle only (∘) (n=4), or 100 ng/ml of MCP-1 (□) (n=1) were injected into the pouches as described in Materials and Methods. Each mark represents the cell number from an individual mouse. After a 4 h incubation, the recruited cells were recovered, counted, and analyzed after cytospin preparation and Wright staining.

FIG. 12 shows that significantly fewer macrophages were recovered from the peritoneal cavity of mice treated with the anti-galectin-3 antibody (α-hu gal3) as compared to mice treated with control antibody (N.S. IgG).

FIG. 13A-13D show immunochemical staining for galectin-3 in the lung tissue and BAL fluid from mice with allergic airway inflammation. (A) H&E staining of a lung section from control, (B) experimental mice, and (C) immunohistochemical staining for galectin-3 of a lung section from control and (D) experimental.

FIG. 14A-14C show detection of galectin-3 in cells and supernatants from BAL fluid. (A) H&E staining of cells and (B) in BAL fluid and immunocytochemical staining for galectin-3 in these cells. C: Three hours after the last antigen challenge, BAL fluid was obtained and galectin-3 levels were determined by ELISA. Each data point represents the mean±SEM of results from three mice; similar results were obtained in three separate experiments.

FIG. 15A-15D show quantitation of leukocyte in BAL fluid from gal3^{+/+} and gal3^{-/-} mice with allergic airway inflammation. (A) BAL fluid was obtained 3 hours after the last challenge and total leukocytes and (B) subpopulations of leukocytes in the fluid were enumerated. The data for neutrophil recoveries are also presented in the inset in B. P values for the differences between gal3^{+/+} and gal3^{-/-} mice: total cells, <0.027; eosinophils, <0.011; macrophages, NS; neutrophils, <0.0278.

FIG. 16A-16B is a comparison of goblet cell mucin production by gal3^{+/+} and gal3^{-/-} mice. A: Representative areas of the lungs from gal3^{+/+} and gal3^{-/-} mice under magnification with ×10 (left) and ×20 (right) objectives in which mucin-producing goblet cells are stained red. B: Comparison of percentages of PAS⁺ goblet cells between gal3^{+/+} and gal3^{-/-} mice (four mice for each genotype). The number of mucin-producing goblet cells in the lungs of gal3^{+/+} mice was significantly higher than in gal3^{-/-} mice (study 1, P<0.014; study 2, P<0.0001).

FIG. 17 show a comparison of AHR between gal3^{+/+} and gal3^{-/-} mice.

FIG. 18A-18D show quantitation of cytokines and immunoglobulin in BAL fluid. Gal3^{+/+} and gal3^{-/-} mice were immunized and then challenged with OVA. The levels of IL-4 (A), IFN-γ (B), total IgE (C), and ratio of OVA-specific IgG_{2α} to IgG₁ (D) in BAL fluid were determined by ELISA. The results represent the mean±SEM of data from a total of 12 mice for each genotype for IL-4 and IgE, 7 mice each for IFN-γ, and 23 mice each for IgG_{2α}/IgG₁. The P values are: IL-4, <0.027; IFN-γ, <0.0227; IgE, <0.05; and IgG_{2α}/IgG₁, <0.014.

FIG. 19A-19B show quantitation of total IgE in sera from gal3^{+/+} and gal3^{-/-} mice. A: Gal3^{+/+} and gal3^{-/-} mice were treated as described in Example 12: The total serum IgE levels were determined by ELISA. The results are the mean±SEM from four experiments with three mice for each genotype in each experiment. P<0.046 for the differences between gal3^{+/+} and gal3^{-/-} mice. B: Gal3^{+/+} and gal3^{-/-} mice were inoculated with 10 μg of OVA in aluminum hydroxide gel intraperitoneally four times on days 0, 14, 21, and 28 and the total IgE levels from sera obtained on days 1, 17, 24, and 32 were determined by ELISA. The arrows indicate the days the mice were immunized. The data are presented as the mean±SEM from one of two studies with four mice for each genotype in each experiment. *, P<0.029; responses between gal3^{+/+} and gal3^{-/-} throughout the entire period are significantly different by analysis of variance (P<0.0242).

### DETAILED DESCRIPTION

The present invention is based on the observation that galectin-3 acts as chemoattractant for monocytes and macrophages. As used herein, “chemoattractant” refers to a substance that elicits accumulation of cells. Similar to many chemoattractants, galectin-3 causes a Ca²⁺ influx in monocytes and both the chemotactic effect and the induction of Ca²⁺ influx involve PTX-sensitive pathway(s). However, cross-desensitization experiments suggest that the signaling pathway(s) appears to be different from that of the presently known chemokine receptors on monocytes. The physiological relevance of the findings is supported by the fact that galectin-3 also selectively recruits monocytes and neutrophils in vivo in a mouse air pouch model.

The finding that galectin-3 is a chemoattractant for macrophages in addition to monocytes is noteworthy, because unlike monocytes, there are few chemokines that have been shown to attract mature macrophages (see Zlotnik et al., Crit. Rev. Immunol. 19:1-47 (1999)). The major monocyte chemoattractant MCP-1, for example, is inactive in this respect. Galectin-3 may be a major factor involved in the influx of macrophages to inflammatory sites. Therefore, galectin-3 may have particular therapeutic utility in attracting macrophages to sites where it would be desirable to increase the presence of this cell type.

Glectin-3-deficient mice develop significantly reduced numbers of peritoneal macrophages compared to wild-type mice when treated with thioglycollate intraperitoneally (Hsu, et al., Am. J. Pathol. 156:1073-83 (2000)). This is highly consistent with the findings of the present invention. Together, these findings suggest that galectin-3 released by the peritoneal cells in thioglycollate-treated mice is responsible, at least in part, for recruiting monocytes and macrophages to the peritoneal cavity. Thus, galectin-3-deficient mice exhibit a lower macrophage response due to the absence of this chemoattractant.

Accordingly, the present invention provides a method for modulating migration of a cell that expresses a galectin-3 receptor comprising contacting the cell with a migration-modulating amount of galectin-3, galectin-3 binding polypeptide, or galectin-3 receptor binding polypeptide. In one embodiment, the invention relates to a method for modulating monocyte, neutrophil or macrophage migration comprising contacting a monocyte, neutrophil or macrophage with a migration-modulating amount of galectin-3, galectin-3 binding polypeptide, or galectin-3 receptor binding polypeptide.

As used herein, “migration modulating-amount” refers to any amount of galectin-3 or galectin-3 binding polypeptide that produces a statistically significant change in the migration of a cell. “Migration” refers to the movement of a cell or group of cells from one location to another. It is intended that migration refer to cell movement resulting from both kinesis (in which the speed or of frequency of cell movement, or cell turning behavior is affected) as well as taxis (in which the direction of cell movement is affected). As demonstrated by the examples described below, cell migration may be modulated according to the present invention both in vitro and in vivo. In vitro migration can be performed, for example, in Boyden chambers. According to one embodiment, migration is modulated in an animal, preferably a mammal, which may be an experimental animal. In one aspect of the invention, the animal is a mouse. In another aspect, the migration may be in a veterinary animal or human, e.g., with a wound, infection, surgical incision, localized or systemic inflammation, tumor or other condition in which it would be desirable to modulate the migration of cells.

Galectin-3 may be produced by any method known in the art. For example, galectin-3 may be purified from cells or tissues normally expressing the polypeptide. Galectin-3 produced by epithelial cells, a major source of this lectin, can contribute to the attraction of monocytes and macrophages during inflammation, and may therefore provide a source of galectin-3 for the methods of the invention. Monocytes and macrophages also produce galectin-3, which may be utilized in the methods of the invention. Any species of animal, including humans, may provide the source material for galectin-3 production, including body fluids such as blood, tissues or cells, including cells expanded using cell culture techniques. The lectin from theses sources may mediate a continued influx of these cell types once the inflammatory process is initiated. Galectin-3 may also be produced by expressing a recombinant galectin-3 polynucleotide in an appropriate host, such as a bacterial, yeast, insect or animal cell. Galectin-3 polynucleotides includes those that are known in the art or functional equivalents or parts of those sequences.

The term “functional” is used herein to refers to any modified version of, for example, a nucleotide or polypeptide which retains the basic function of its unmodified form. As an example, it is well-known that certain alterations, mutations or polymorphisms in amino acid or nucleic acid sequences may not affect the polypeptide encoded by that molecule or the function of the polypeptide. It is also possible for deleted versions of a molecule to perform a particular function as well as the original molecule. Even where an alteration does affect whether and to what degree a particular function is performed, such altered molecules are included within the term “functional equivalent” provided that the function of the molecule is not so deleteriously affected as to render the molecule useless for its intended purpose, particularly modulating cell migration.

According to the methods of the invention, migration of cells, including monocytes, neutrophils and macrophages, can be modulated, that is stimulated, inhibited or directed. Recombinant human galectin-3 induces monocyte migration in vitro and it is chemotactic at high concentrations (1.0 μM) but chemokinetic at low concentrations (10-100 nM). As used herein, “chemokinetic” refers to a response by a motile cell to a substance that involves an increase or decrease in speed or frequency of movement or a change in the frequency or magnitude of turning behavior. In contrast, “chemotactic” refers to a response of motile cells in which the direction of movement is affected by the substance. Chemotaxis differs from chemokinesis in that the substance alters probability of motion in one direction only, rather than rate or frequency of random motion in all directions.

The skilled artisan will recognize that the amount of galectin-3, galectin-3 binding polypeptide, or galectin-3 receptor binding polypeptide required to produce a change or modulation in the migration of a cell will depending on the type of cell modulated, the context of that cell (e.g., in vitro versus in vivo; tumor versus wound), and the qualitative change in migration desired. For example, the amount of galetcin-3 required to inhibit cell migration may be different than that required to stimulate cell migration. Similarly, the amount required to reduce generalized cell migration in systemic inflammation may be different than that required to topically enhance cell migration to a localized site of tissue injury.

It has been shown previously that galectin-3 can activate various cell types including induction of superoxide production by monocytes/macrophages (Liu, et al., Am. J. Pathol. 147:1016-29 (1995)). Although the precise mechanisms of action still remain to be determined, these activities are probably related to the dimerization or oligomerization of galectin-3 through intermolecular interactions involving the amino-terminal domain (Hsu, et al., J. Biol. Chem. 267:14167-74 (1992)). The lectin thereby becomes bivalent or multivalent functionally and capable of activating cells by effectively crosslinking cell-surface glycoproteins (Barondes, et al., J. Biol. Chem. 269:20807-10 (1994); Kasai, et al., J. Biochem. (Tokyo) 119:1-8 (1996); Perillo, et al., J. Mol. Med. 76:402-12 (1998); Hughes, Biochem. Soc. Trans. 25:1194-2298 (1997); Liu, Immunol. Today 14:486-90 (1993)). This process may also contribute to the monocyte chemoattractant activity of galectin-3 and this possibility is supported by the finding of the present invention that both the N-terminal and C-terminal domains of galectin-3 are required for this activity. However, an unusual feature of galectin-3's chemoattractant activity is that the response is both qualitatively and quantitatively dependent on the concentration of the lectin. First, galectin-3 is chemokinetic at low concentrations but chemotactic at high concentrations. One possible explanation is that galectin-3 at high concentrations can cause cell aggregation, and, thus, in the checkerboard analysis (described below), when galectin-3 is added to the upper chambers together with the cells, the cells are prevented from migrating towards the lower chambers because they are aggregated. Therefore, it is possible that galectin-3 is actually chemokinetic for monocytes at both high and low concentrations.

However, only monocyte migration induced by high concentrations of galectin-3 is inhibited by PTX. Also, only high concentrations of galectin-3 caused a Ca²⁺ influx in monocytes and this occurred through a PTX-sensitive mechanism(s). The most likely explanation for these findings is that galectin-3 binds to and activates different (or different sets of) cell surface molecules depending on its concentration. At lower concentrations, it preferentially binds to glycoproteins that interact with the lectin relatively strongly, while only after reaching a certain threshold concentration, it begins to recognize other cell surface glycoproteins that interact with the lectin relatively weakly. The latter may include PTX-sensitive G-protein coupled receptor(s). Galectin-3 has been shown to bind to a number of different cell surface glycoproteins on macrophages (Dong and Hughes, Glycoconjugate J. 14:267-74 (1997) and, based on a recent study with galectin-1 (Pace, et al., J. Immunol. 163:3801-11 (1999), it is likely that the lectin can cause segregation of these different glycoproteins. It is entirely possible that the lectin binds to these different glycoproteins with variable affinity, because they are differentially glycosylated and the lectin exhibits a fine specificity to oligosaccharides (Sparrow, et al., J. Biol. Chem. 262:7383-90 (1987); Leffler and Barondes, J. Biol. Chem. 261:10119-26 (1986); Feizi, Biochemistry 33:6342-49 (1994)).

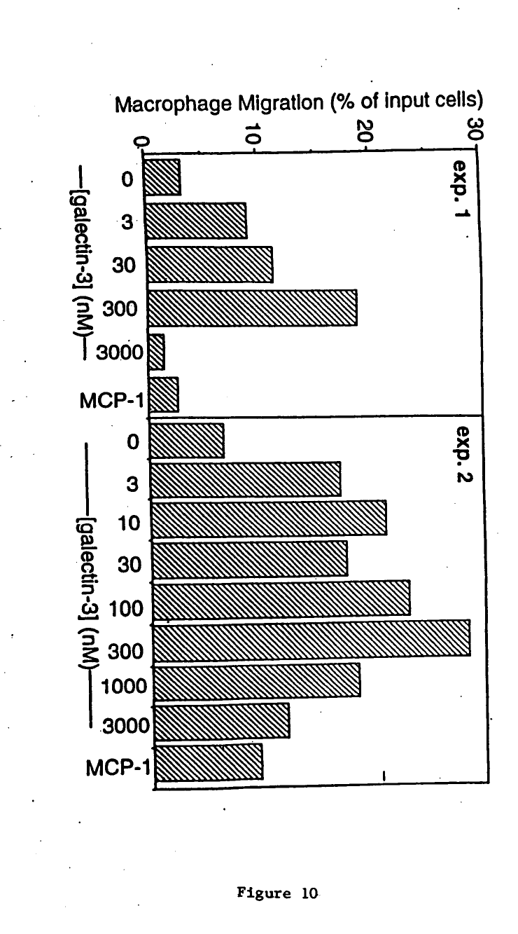

Relatively high concentrations of galectin-3 are needed for the demonstration of optimal experimental chemoattractant activity. The situation is analogous to other activities demonstrated for this lectin previously, such as activation of inflammatory cells (Liu, et al., Am. J. Pathol. 147:1016-29 (1995); Frigeri, et al., Biochemistry 32:7644-49 (1993); Yamaoka, et al., J. Immunol. 154:3479-87 (1995)), and is probably related to the concentrations that are required for the dimerization or oligomerization of the lectin to take place. However, galectin-3 is known to exist at relatively high concentrations in the cytosol of many cell types (e.g., 5 μM in a human colon adenocarcinoma cell line, T84 (Huflejt, et al., J. Biol. Chem. 272:14294-303 (1997)). Therefore, a high local concentration of the lectin may be achieved when there is a burst release of the protein from these cells. In fact, galectin-3 has been found to be present in significant amounts in biological fluids. For example, the concentrations of galectin-3 in bronchoalveolar lavage fluid from mice with airway inflammation were found to be over 20 nM. Considering the dilution factor introduced in obtaining the lavage fluid, it is easily conceivable that the initial local concentrations of the lectin are in the micromolar range. On the other hand, the effective concentrations of galectin-3 for attracting alveolar macrophages are much lower (FIG. 10), approaching those typically found for many chemokines. It is possible that the putative receptor for galectin-3 on these cells either exists in higher numbers or interacts with the lectin more strongly. Alternatively, the putative receptor on these cells transmits signals more effectively upon interacting with the lectin.

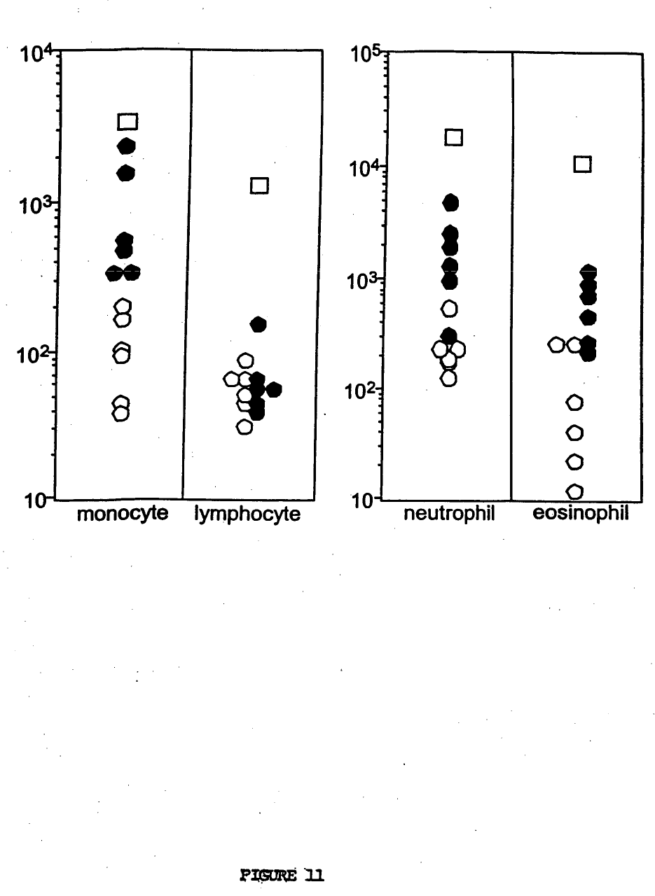

Galectin-3 probably activates PTX-sensitive G-protein-coupled receptors similar to those recognized by many known chemokines (Baggiolini, Nature 392:565-68 (1998); Sallusto, et al., Immunol. Today 19:568-74 (1998)). This lectin does not have significant sequence similarity with any of these chemokines, and thus it appears unlikely that it recognizes these receptors through protein-protein interactions, but it could do so via lectin-carbohydrate interactions. Chemokine receptors expressed on monocytes include CCR-1, CCR-2, CCR-5, and CXCR-4 (Baggiolini, Nature 392:565-68 (1998); Sozzani, et al., J. Immunol. 150:1544-53 (1993)); Bizzari, et al., Blood 86:2388-94 (1995); Oberlin, et al., Nature 382:833-35 (1996); Sallusto, et al., Immunol. Today 19:568-74 (1998)). However, no cross-desensitization has been observed between galectin-3 and any of the monocyte-reactive chemokines that utilize these receptors, including MCP-1 for CCR-2, MIP-1α for CCR-1 and CCR-5, and SDF-1α for CXCR-4. Neither have interactions between galectin-3 and these four chemokine receptors been detected by immunoprecipitation and immunoblotting using specific antibodies. It has been reported that CCR-3 may be also expressed on human monocytes and macrophages (Fantuzzi, et al., Blood 94:875-83 (1999)). However, the usage of this receptor was not analyzed because galectin-3 does not attract eosinophils (which are known to express CCR-3) in vitro (not shown) or in vivo (FIG. 11), suggesting no interaction of galectin-3 with this receptor. Therefore, although the precise receptor for galectin-3 remains undetermined, it is not any of the known receptors, such as CCR-1, CCR-2, CCR-3, CCR-5 and CXCR-4.

Other types of chemoattractant receptors, including those for N-formyl-Met-Leu-Phe (fMLP), platelet activating factor (PAF), leukotrienes, and C5a, could mediate the effects of galectin-3. Galectin-3 is also known to recognize CD11b, LAMPs1 and 2, Mac-3, and CD98 on thioglycollate-stimulated mouse peritoneal macrophages (Dong and Hughes, Glycoconjugate J. 14:267-74 (1997). Stimulation and/or cross-linking of CD11b and CD98 could enhance adhesion and transendothelial migration of monocytes (Meerschaert and Furie, J. Immunol. 154:4099-112 (1995); Fenczik, et al., Nature 390:81-85 (1997)).

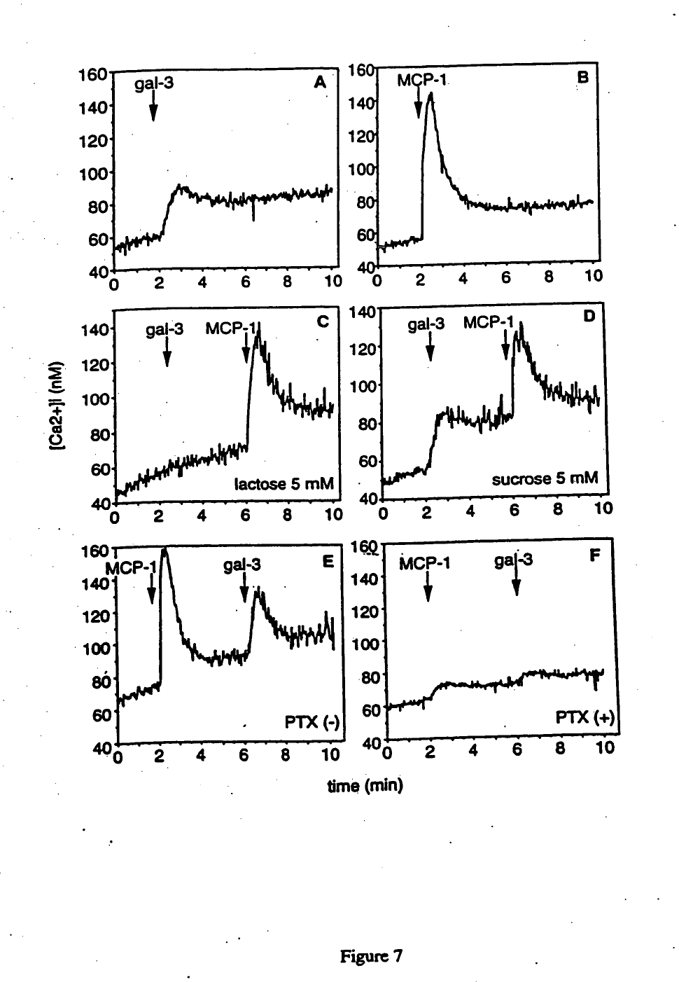

According to the methods of the invention, the cell type modulated may be any cell type that expresses a galectin-3 receptor and for which galectin-3 has an effect upon cell migration. It is to be noted that while galectin-3 is likely to bind to a number of different cell types through lectin-carbohydrate interactions, its chemoattractant activity is cell-type specific, as it does not induce migration of lymphocytes in vitro, or in vivo as shown in FIG. 11. This selectivity could be explained by the differential expression of the putative galectin-3 receptor on different cell types. For example, galectin-3 is known to cause a Ca²⁺ influx in Jurkat T cells, but the effect was sustained and insensitive to PTX (Dong and Hughes, FEBS Lett. 395:165-69 (1996), in contrast to the case in monocytes (FIG. 7). Thus, this lectin can use different receptors on different cell types, resulting in the activation of selected types of cells, or causing a similar effect(s) on different types of cells by alternative pathways. Furthermore, galectin-3 may be a chemoattractant for neutrophils and eosinophils as well. Lower concentrations of this lectin were required for maximum migration of neutrophils compared with monocytes. In addition, galectin-3-induced recruitment of neutrophils in the mouse air pouch experiments (FIG. 11) and. The neutrophil chemoattractant activity of galectin-3 is also consistent with the results obtained from studies of galectin-3-deficient mice by other investigators (Colnut, et al., Immunol. 94:290-96 (1998)), who noted that galectin-3 deficiency results in a significantly lower degree of neutrophil response in the peritoneal cavity following thioglycollate stimulation.

Galectin-3 may also play an important role in the function of mast cells. Bone marrow-derived mast cells (BMMC) from wild type [gal-3 (+/+)] and galectin-3 deficient [gal-3 (−/−)] mice show comparable expression of IgE receptor and c-kit. However, upon activation by both FceRI cross-linking and calcium ionophore stimulation, gal-3 (−/−) BMMC secrete a less histamine, b-hexosaminidase and pro-inflammatory cytokine TNF- than gal-3 (+/+) BMMC. Gal-3 (−/−) BMMC grow poorly in culture as compared to gal-3 (+/+) BMMC, suggesting that galectin-3 may be involved in the regulation of apoptosis of mast cells. When these cells are deprived of growth factors, apoptosis is differentially induced: more apoptosis is observed in 3-week old gal-3 (−/−) BMMC than in gal-3 (+/+) BMMC. However, 4-week old gal-3 (−/−) BMMC are more resistant to apoptosis, suggesting a that there is a defect in signal transduction in gal-3 (−/−) BMMC. Further support for this conclusion is found in the strikingly lower basal level of c-jun-N-terminal kinase (JNK) in cell lysates from gal-3 (−/−) BMMC than in gal-3 (+/+) BMMC (as detected by immunoblotting). In contrast, comparable levels of several other kinases are detectable in the cell lysates from the two genotypes. Further, our results show that JNK is inducible in vitro in both gal-3 (+/+) and gal-3 (−/−) BMMC upon FceRI cross-linking, but immunoprecipitates from gal-3 (−/−) BMMC have significantly reduced ability to phosphorylate the JNK substrate c-jun in an in vitro kinase assay.

In one aspect of the invention, the galectin-3 comprises an N-terminal or C-terminal subsequence of galectin-3. Both the N-terminal and C-terminal domains of galetin-3 appear to be involved in the migration-modulation activity, which can be inhibited by either lactose or the C-terminal domain fragment. Specific monoclonal antibody to galectin-3 was found to inhibit the activity. Thus, the methods of the invention can be practiced using a galectin-3 binding protein, such as a galectin-3 antibody or binding fragment thereof.

As used herein, the term “antibody” refers to intact antibody molecules as well as fragments thereof, such as Fab, F(ab′)₂, Fv and scFv fragments, which are capable of binding the epitopic determinant. “Antibody” refers to any polyclonal or monoclonal immunoglobulin molecule, such as IgM, IgG, IgA, IgE, IgD, and any subclass thereof, such as IgG₁, IgG₂, IgG₃, IgG₄, etc. The term “antibody” also means a functional fragment or subsequence of immunoglobulin molecules, such as Fab, Fab′, F(ab′)₂, Fv, Fd, scFv and sdFv, unless otherwise expressly stated.

Galectin-3 antibodies include antibodies having either or both of antibody-dependent cell-mediated cytotoxicity (ADCC) and complement-mediated cytotoxicity (CDC) activities. IgG subclass IgG₁ is known to exhibit both ADCC and CDC activities.

The terms “galectin-3 antibody” or “anti-galectin-3 antibody” means an antibody that specifically binds to galectin-3 protein. Specific binding is that which is selective for an epitope present in galectin-3. Thus, binding to proteins other than galectin-3 is such that the binding does not significantly interfere with detection of galectin-3 or galectin-3 subsequences, unless such other proteins have a similar or the same epitope present in galectin-3 protein so as to be recognized by galectin-3 antibody. Selective binding can be distinguished from non-selective binding using assays known in the art.

Human, humanized and primarized antibodies are also contemplated by the present invention. The term “human” when used in reference to an antibody, means that the amino acid sequence of the antibody is fully human. A “human galectin-3 antibody” or “human anti-galectin-3 antibody” therefore refers to an antibody having human immunoglobulin amino acid sequences, i.e., human heavy and light chain variable and constant regions that specifically bind to galectin-3. That is, all of the antibody amino acids are human or exist in a human antibody.

An antibody that is non-human may be made fully human by substituting the non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); and Chothia and Lesk J. Mol. Biol. 186:651 (1987)). A consensus sequence of human V_{H} subgroup III, based on a survey of 22 known human V_{H} III sequences, and a consensus sequence of human V_{L} kappa-chain subgroup I, based on a survey of 30 known human kappa I sequences is described in Padlan Mol. Immunol. 31:169 (1994); and Padlan Mol. Immunol. 28:489 (1991)).

The term “humanized antibody”, as used herein, refers to antibody molecules in which amino acids have been replaced in the non-antigen binding regions in order to more closely resemble a human antibody, while still retaining the original binding ability. The term “humanized” therefore means that the amino acid sequence of the antibody has non-human amino acid residues (e.g., mouse, rat, goat, rabbit, etc.) of one or more determining regions (CDRs) that specifically bind to the desired antigen (e.g., galectin-3) in an acceptor human immunoglobulin molecule, and one or more human amino acid residues in the Fv framework region (FR), which are amino acid residues that flank the CDRs. Human framework region residues of the immunoglobulin can be replaced with corresponding non-human residues. Residues in the human framework regions can therefore be substituted with a corresponding residue from the non-human CDR donor antibody to alter, generally to improve, antigen affinity or specificity, for example. In addition, a humanized antibody may include residues, which are found neither in the human antibody nor in the donor CDR or framework sequences. For example, a framework substitution at a particular position that is not found in a human antibody or the donor non-human antibody may be predicted to improve binding affinity or specificity human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., Nature 332:323 (1988)). Antibodies referred to as “primatized” in the art are within the meaning of “humanized” as used herein, except that the acceptor human immunoglobulin molecule and framework region amino acid residues may be any primate residue, in addition to any human residue.

An exemplary antibody is denoted B2C10. Antibody B2C10, as well as antibodies having the binding specificity of B2C10 may be used in accordance with the invention compositions and methods. Antibodies that bind to an amino acid sequence to which B2C10 galectin-3 antibody binds also may be used in accordance with the invention compositions and methods.

The term “binding specificity,” when used in reference to an antibody, means that the antibody specifically binds to all or a part of the same antigenic epitope or sequence as the reference antibody. Thus, a galectin-3 antibody having the binding specificity of B2C10 specifically binds to all or a part of the same epitope or sequence as the galectin-3 antibody denoted B2C10. A part of an antigenic epitope or sequence means a subsequence or a portion of the epitope or sequence. For example, if an epitope includes 8 contiguous amino acids, a subsequence and, therefore, a part of an epitope may be 7 or fewer amino acids within this 8 amino acid sequence epitope. In addition, if an epitope includes non-contiguous amino acid sequences, such as a 5 amino acid sequence and an 8 amino acid sequence which are not contiguous with each other, but form an epitope due to protein folding, a subsequence and, therefore, a part of an epitope may be either the 5 amino acid sequence or the 8 amino acid sequence alone.

Epitopes typically are short amino acid sequences, e.g. about five to 15 amino acids in length. Systematic techniques for identifying epitopes are also known in the art and are described, for example, in U.S. Pat. No. 4,708,871. Briefly, a set of overlapping oligopeptides derived from galectin-3 may be synthesized and bound to a solid phase array of pins, with a unique oligopeptide on each pin. The array of pins may comprise a 96-well microtiter plate, permitting one to assay all 96 oligopeptides simultaneously, e.g., for binding to an anti-galectin-3 monoclonal antibody. Alternatively, phage display peptide library kits (New England BioLabs) are currently commercially available for epitope mapping. Using these methods, binding affinity for every possible subset of consecutive amino acids may be determined in order to identify the epitope that a particular antibody binds. Epitopes may also be identified by inference when epitope length peptide sequences are used to immunize animals from which antibodies that bind to the peptide sequence are obtained.

Galectin-3 antibodies also include human, humanized and chimeric antibodies having the same binding affinity and having substantially the same binding affinity as the galectin-3 antibody B2C10. For example, a galectin-3 antibody may have an affinity greater or less than 2-5, 5-10, 10-100, 100-100 or 1000-10,000 fold affinity as the reference galectin-3 antibody. Typical antibody affinities for galectin-3 have a dissociation constant (Kd) less than 5×10⁻⁴ M, 10⁻⁴ M 5×10⁻⁵ M, 10⁻⁵ M 5×10⁻⁶ M, 10⁻⁶ M 5×10⁻⁷ M, 10⁻⁷ M 5×10⁻⁸ M, 10⁻⁸ M 5×10⁻⁹ M, 10⁻⁹ M 5×10⁻¹⁰ M, 10⁻¹⁰ M 5×10⁻¹¹ M, 10⁻¹¹ M 5×10⁻¹² M, 10⁻¹² M 5×10⁻¹³ M, 10⁻¹³ M 5×10⁻¹⁴ M, 10⁻¹⁴ M 5×10⁻¹⁵ M, and 10⁻¹⁵ M.

As used herein, the term “the same,” when used in reference to antibody binding affinity, means that the dissociation constant (K_{D}) is within about 5 to 100 fold of the reference antibody (5-100 fold greater affinity or less affinity than the reference antibody). The term “substantially the same” when used in reference to antibody binding affinity, means that the dissociation constant (K_{D}) is within about 5 to 5000 fold of the reference antibody (5-5000 fold greater affinity or less affinity than the reference antibody).

Methods for producing both polyclonal and monoclonal antibodies are well known in the art (see, for example, Harlow and Lane, Antibodies: A Laboratory Manual, Cold Spring Harbor Laboratory (1988)). Antibodies that bind galectin-3 can be prepared, for example, using intact polypeptides or fragments containing small peptides of interest as the immunizing antigen. The polypeptides or peptides used to immunize an animal can be derived for example, from protein isolated from cells or tissues, by translation of mRNA or synthesized chemically, and can be conjugated to a carrier protein, if desired. Commonly used carriers that are chemically coupled to peptides include bovine serum albumin and thyroglobulin. The coupled peptide is then used to immunize the animal (e.g., a mouse, rabbit, rat, sheep, goat, cow, or guinea pig). Additionally, to increase the immune response, galectin-3 can be coupled to another protein such as ovalbumin or keyhole limpet hemocyanin (KLH), thyroglobulin and tetanus toxoid, or mixed with an adjuvant such as Freund's complete or incomplete adjuvant. Initial and any optional subsequent immunization may be through intraperitoneal, intramuscular, intraocular, or subcutaneous routes. Subsequent immunizations may be at the same or at different concentrations of galectin-3 preparation, and may be at regular or irregular intervals.

Methods of producing human antibodies are known in the art. For example, human transchromosomic KM mice™ (WO 02/43478) and HAC mice (WO 02/092812). express human immunoglobulin genes. Using conventional hybridoma technology, splenocytes from immunized mice that respond to galectin-3 can be isolated and fused with myeloma cells. An overview of the technology for producing human antibodies is described in Lonberg and Huszar, Int. Rev. Immunol. 13:65 (1995). Transgenic animals with one or more human immunoglobulin genes (kappa or lambda) that do not express endogenous immunoglobulins are described, for example in, U.S. Pat. No. 5,939,598. Additional methods for producing human antibodies and human monoclonal antibodies are described (see, e.g., WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Galectin-3 monoclonal antibodies can also be readily generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988). Suitable techniques that additionally may be employed in the method including affinity purification, non-denaturing gel purification, HPLC or RP-HPLC, purification on protein A column, or any combination of these techniques. The antibody isotype can be determined using an ELISA assay, for example, a human Ig can be identified using mouse Ig-absorbed anti-human Ig.

Antibodies can be humanized using a variety of techniques known in the art including, for example, CDR-grafting (EP 239,400; W091/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan Mol. Immunol. 31:169 (1994); and Padlan Mol. Immunol. 28:489 (1991)) have previously used to humanize antibodies (Carter et al. Proc. Natl. Acad. Sci. USA 89:4285 (1992); and Presta et al. J. Immunol. 151:2623 (1993)).

Methods for producing chimeric antibodies are known in the art (e.g., Morrison, Science 229:1202 (1985); Oi et al., BioTechniques 4:214 (1986); Gillies et al., (1989) J. Immunol. Methods 125:191; and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Antibodies according to the present invention also include recombinant antibody molecules, or fragments thereof, expressed from cloned antibody-encoding polynucleotides, such as polynucleotides isolated from hybridoma cells or selected from libraries of naturally occurring or synthetic antibody genes (see for example, Gram et al., Proc. Natl. Acad. Sci. USA 89:3576-80 (1992)).

The skilled artisan will recognize that galectin-3 receptor binding polypetides may have the same effect as galectin-3 by acting as agonists of galectin-3 receptors. Polypeptides that bind galectin-3 receptors may also behave as antagonists, thereby competing with galectin-3. Both types of galectin-3 receptor binding polypeptides may be used to modulate migration of a cell and are therefore within the scope of this invention. Polypeptides can range from about 10-20, 20-30, 30-40, 40-50, 50-60, 60-75, 75-100, 100-150, 150-200 or more amino acid residues, up to the full length native sequence.

Exemplary inhibitors of galectin-3 activity include galectin-3 subsequences that retain carbohydrate-binding activity; N-terminal and C-terminal subsequences of galectin-3. Exemplary peptides that function as galectin 3 include, for example,

SMEPALPDWWWKMFK; DKPTAFVSVYLKTAL; PQNSKIPGPTFLDPH;
APRPGPWLWSNADSV; GVTDSSTSNLDMPHW; PKMTLQRSNIRPSMP;
PQNSKIPGPTFLDPH; LYPLHTYTPLSLPLF;
LTGTCLQYQSRCGNTR; AYTKCSRQWRTCMTTH;
ANTPCGPYTHDCPVKR; NISRCTHPFMACGKQS; and
PRNICSRRDPTCWTTY.

Inhibitors of galectin-3 further include galactose and derivatives thereof. Non-limiting examples of galactose derivative include galactosides, such as thio-galactoside and a thiodi-galactosides.

Specific exemplary thio-galactosides and thiodi-galactosides include:

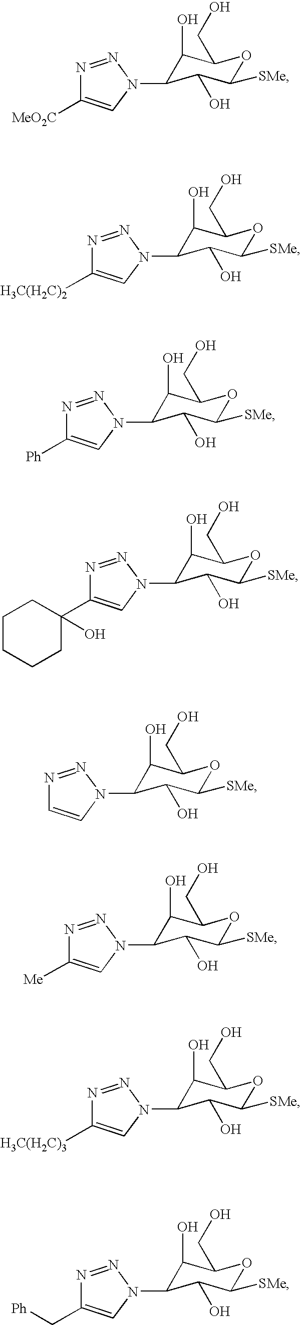

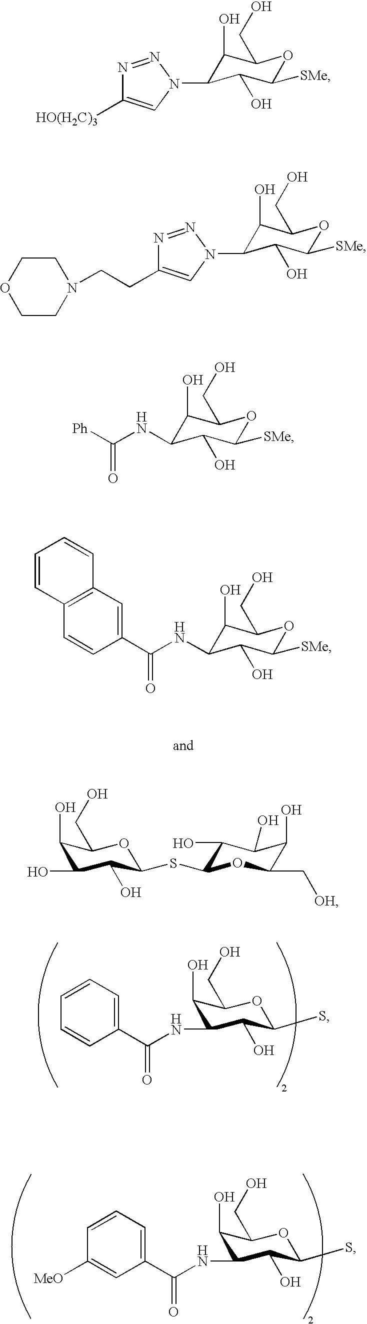

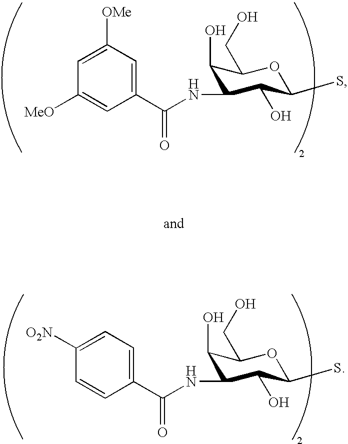

Additional inhibitors of galectin-3 activity include glycoconjugates, or derivatived that binds galectin-3. Non-limiting examples include glycolipids, glycopeptides and proteoglycans. Exemplary glycolipids are as set forth in Table A. Exemplary glycopeptides are as set forth in Table B.

Further inhibitors of galectin-3 activity include saccharides (e.g., monosaccharides, di-saccharide, tri-saccharide, polysaccharaides and oligosaccharides). Saccharides include lactose, tetrasaccharide, beta-galactoside, as well as analogs and derivatives thereof, which may be naturally occurring or synthetic. Exemplary saccharides include, for example, lactose; Galβ1,4GlcNAcβ1,3Galβ1,4Glc; Galβ1,3GlcNAcβ1,3Galβ1,4Glc; PNP βLacNAc; PNP βGalβ1,3GlcNAc; Galβ1,4GlcNAcβ1,3Gal; LacNAc; Galβ1,4GlcNAcβ1,2(Galβ1,4GlcNAcβ1,6)Man; MeβLacNAc; Galβ1,4GlcNAcβ1,2(Galβ1,4GlcNAcβ1,4)Manα1,3)(Galβ1,4GlcNAcβ1,2(Galβ1,4GlcNAcβ1,6)Manα1,6)Man; Galβ1,4Fru; Galβ1,4ManNAc; Galα1,6Gal; MeβGal; GlcNAcβ1,3Gal; GlcNAcβ1,4GlcNAc; Glcβ1,4Glc; and GlcNAc. Exemplary oligosaccharides include, for example, compounds set forth in Table B.

Yet additional inhibitors of galectin-3 activity include glycodendrimers. Exemplary glycodendrimers include, for example:

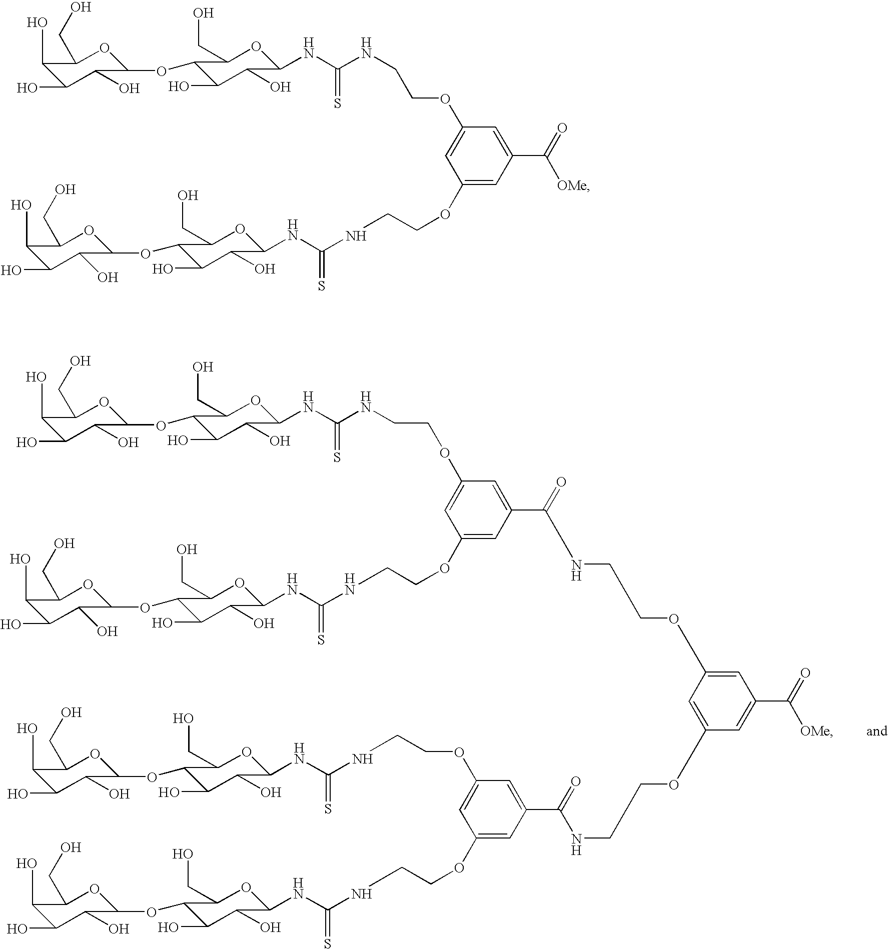

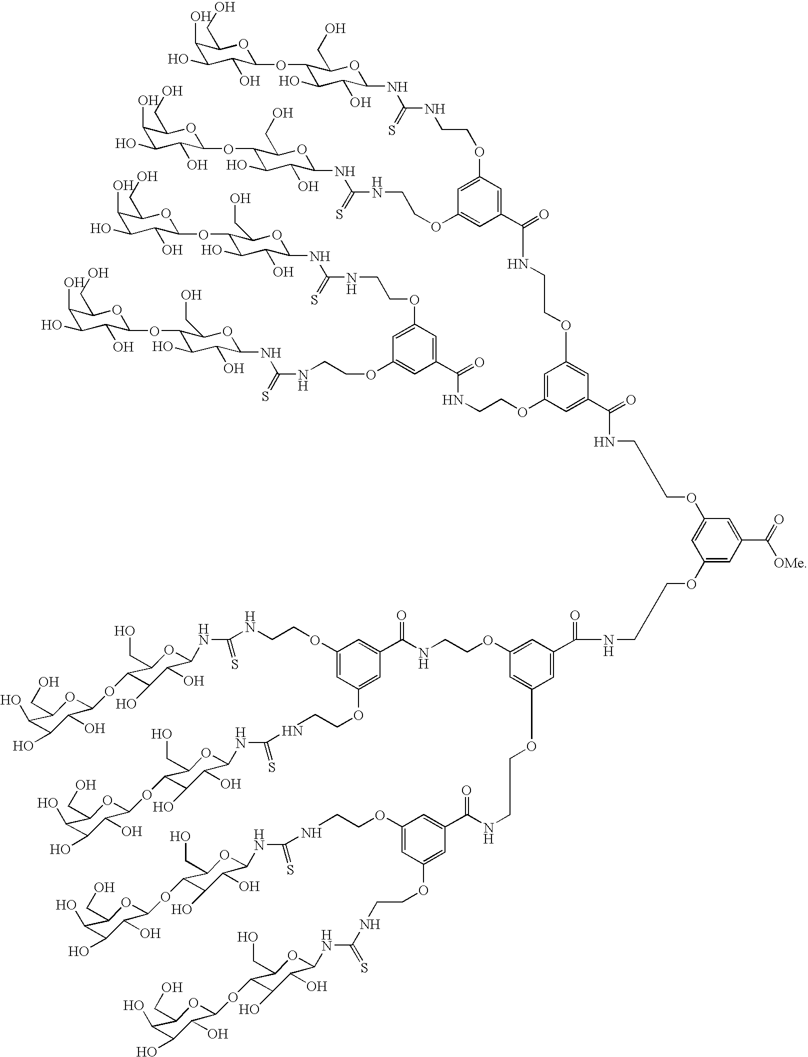

Still additional inhibitors of galectin-3 activity include N-acetyl lactosamine, and derivatives thereof. N-acetyl lactosamine derivatives include a C3′ amides, sulfonamides and urea derivatives. Exemplary C3′ amides include, for example:

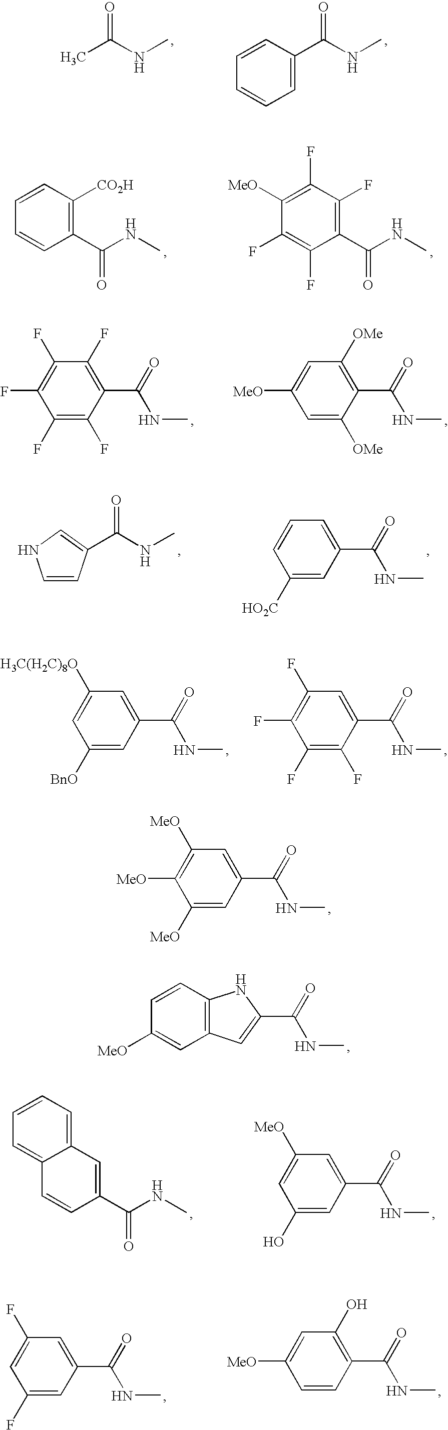

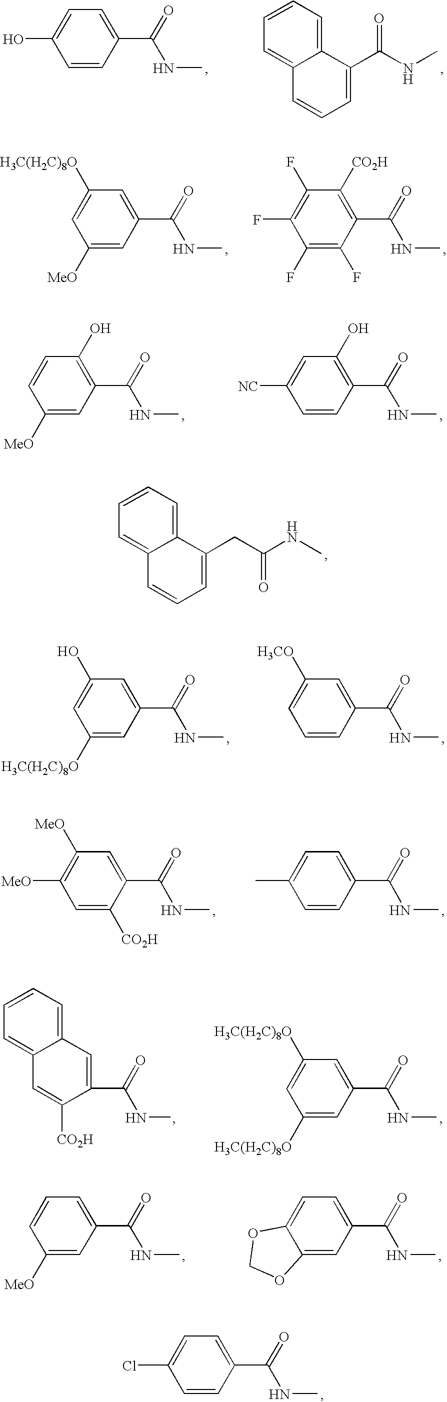

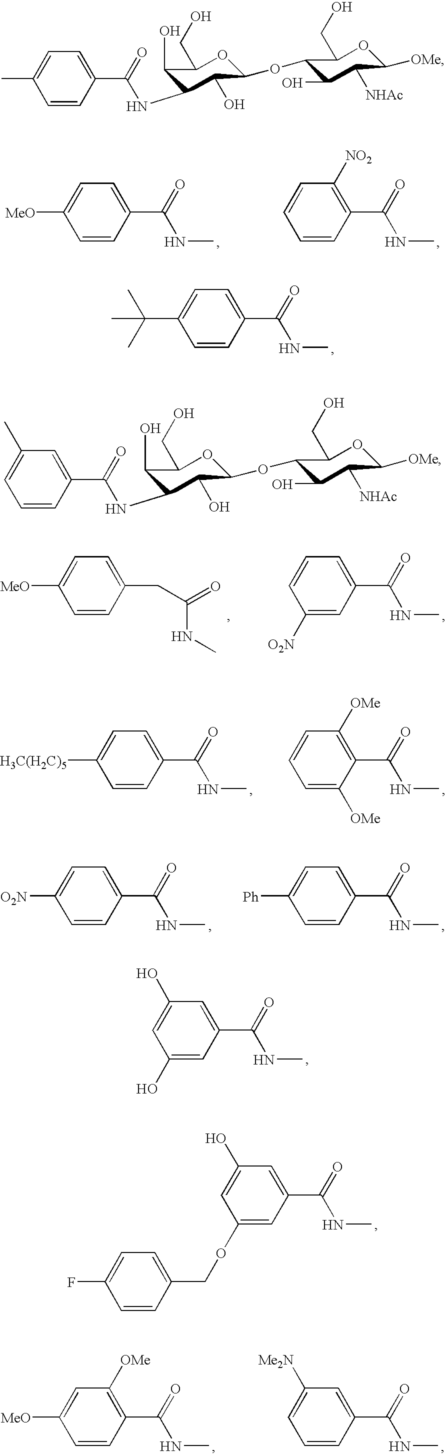

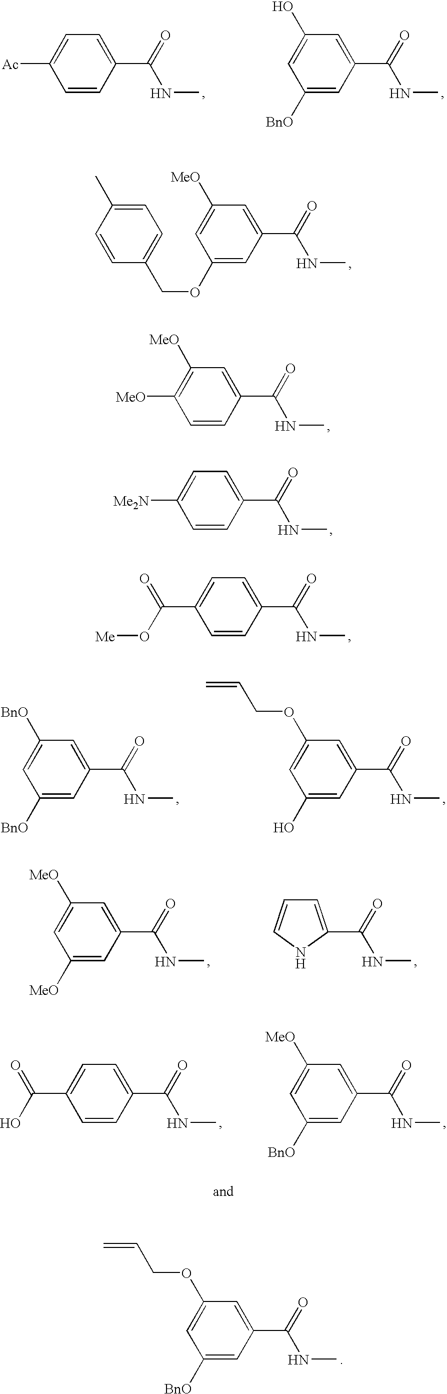

TABLE A

| Designation | Sequance |
|---|---|
| pLNnP | GlcNAcβ1-3Galβ1-4GlcNAcβ1-3Galβ1-4Glc |
| pLNnH | Galβ1-4GlcNAcβ1-3Galβ1-4GlcNAcβ1-3Galβ1-4Glc |
| pLNH | Galβ1-3GlcNAcβ1-3Galβ1-4GlcNAcβ1-3Galβ1-4Glc |
| LNFP-I | 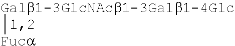 |
| Cer 5 | Galα1-3Galβ1-4GlcNAcβ1-3Galβ1-4Glc |
| A6 | 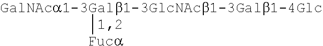 |
| Cer B6 | 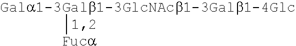 |
| S1 | NeuAcα2-3Galβ1-3GlcNAcβ1-3Galβ1-4Glc |
| Cer 8 | 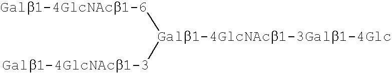 |
| Cer 10 | 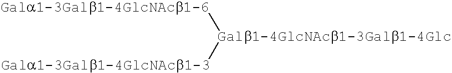 |
| Cer 12 | 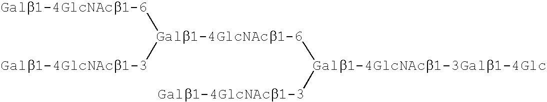 |
| Cer 15 | 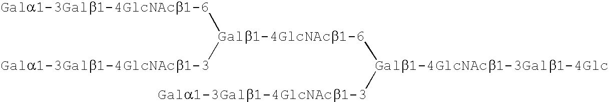 |
| GM₁-A | 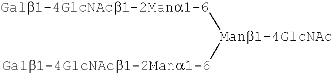 |
| GM₁-B | 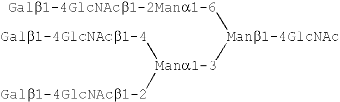 |
| GM₁-C | 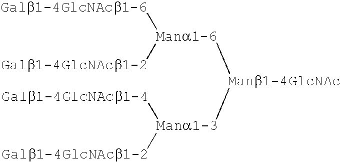 |
| Lac/Lac Cer | Galβ1-4Glc |
| LNT | Galβ1-3GlcNAcβ1-3Galβ1-4Glc |
| LNnT or Cer 4 | Galβ1-4GlcNAcβ1-3Galβ1-4Glc |
| Cer 9 | 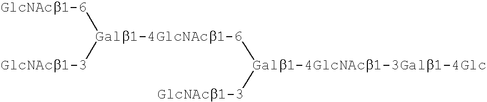 |
| LNFP-II | 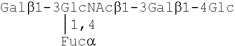 |
| LNFP-III | 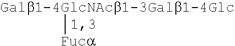 |
| LNDFH-I | 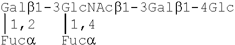 |
| A2 | GalNAcα1-3Gal |
| A3 | 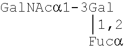 |
| A4 | 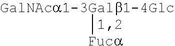 |
| A5 | 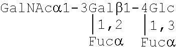 |
| A7 | 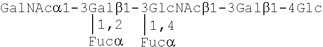 |
| S3 | NeuAcα2-6Galβ1-4GlcNAcβ1-3Galβ1-4Glc |
| As GN₂ Cer | GalNAcβ1-4Galβ1-4Glc |
| As GN₁ Cer | Galβ1-3GalNAcβ1-4Galβ1-4Glc |
| GN₂ Cer | 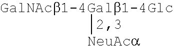 |
| GN₁ Cer | 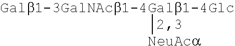 |
| BGN₃ Cer | 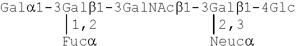 |
| Globoside Cer | GalNAcβ1-3Galα1-4Galβ1-4Glc |
| Forssman Cer | GalNAcα1-3GalNAcβ1-3Galα1-4Galβ1-4Glc |
| GN₃ | GlcNAcβ1-4GlcNAc |

TABLE B

| Number | Formula^{a} |
|---|---|
| 1 | Galβ-4Glc |
| 2 | 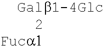 |
| 3 | 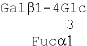 |
| 4 | 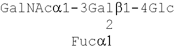 |
| 5 | Galα1-4Galβ1-4GlcβOMe |
| 6 | GalNAcβ1-4Galβ1-4Glc |
| 7 | NeuAcα2-3Galβ1-4Glc |
| 8 | NeuAcα2-6Galβ1-4Glc |
| 9 | GalNAcβ1-3Galα1-4Galβ1-4Glc |
| 10 | Galβ1-4Fru |
| 11 | Thiodigalactoside (Galβ1-S-1βGal) |
| 12 | Galβ1-4GlcNAc |
| 13 | Galβ1-3GlcNAc |
| 14 | Galβ1-3GalNAc |
| 15 | GalNAcβ1-3GalαOMe |
| 16 | Galα1-3GalαOMe |
| 17 | GlcNAcβ1-3GalβOMe |
| 18 | Galα1-4Gal |
| 19 | Glcβ1-4Glc |
| 20 | Galβ1-3GlcNAcβ1-3Galβ1-4Glc |
| 21 | 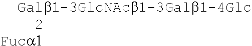 |
| 22 | 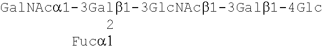 |
| 23 | 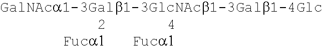 |
| 24 | Asialofetuin oligosaccharide |
| 25 | Fetuin oligosaccharide |
| 26 | Asialoorosomucoid oligosaccharide |
| 27 | Orosomucoid oligosaccharide |
| 28 | Granulocyte LAG glycopeptide |
| 29 | Cord erythrocyte LAG glycopeptide |
| 30 | Adult erythrocyte LAG glycopeptide |
| 31 | Adult erythrocyte LAG |
| 32 | 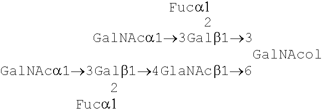 |
| 33 | 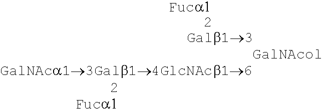 |

Additional inhibitors of galectin-3 include nucleic acid, such as “antisense,” which refers to a polynucleotide or peptide nucleic acid capable of binding to a specific DNA or RNA sequence. Such antisense can inhibit galectin-3 expression. Such antisense can be made by producing a polynucleotide targeted to all or a region of galectin-3 (e.g., 5′ or 3′ untranslated region, intron or gene coding region) and testing for inhibition of galectin-3 expression, for example, in a cell that expresses galectin-3.

Antisense includes single, double or triple stranded polynucleotides and peptide nucleic acids (PNAs) that bind RNA transcript or DNA. For example, a single stranded nucleic acid can target galectin-3 transcript (e.g., mRNA). Oligonucleotides derived from the transcription initiation site of the gene, e.g., between positions −10 and +10 from the start site, are a particular one example. Triplex forming antisense can bind to double strand DNA thereby inhibiting transcription of the gene. The use of double stranded RNA sequences (known as “RNAi”) for inhibiting gene expression is known in the art (see, e.g., Kennerdell et al., Cell 95:1017(1998); Fire et al., Nature, 391:806(1998)). Double stranded RNA sequences from a galectin-3 coding region may therefore be used to inhibit expression.

The methods of the present invention may be useful in therapeutic applications where it is desirable to increase or decrease the number or rate of migration of cells, particularly migration of cells of the immune system to the site of inflammation, infection or a tumor. “Infection” as used herein, refers to the invasion and multiplication of foreign microorganisms such as bacteria, fungi including yeast, viruses and the like, in body tissues of a host organism, particularly a human. Infections may be unapparent, but frequently are harmful to the normal functioning of the host organism, resulting in local cellular injury due to competitive metabolism, toxins, intracellular replication or antigen-antibody response. The infection may remain localised, subclinical and temporary if the body's defensive mechanisms are effective. A local infection may persist and spread by extension to become an acute, subacute or chronic clinical infection or disease state. A local infection may also become systemic when the microorganisms gain access to the lymphatic or vascular system.

The term “inflammation” as used herein, is a pathologic process of cytologic and chemical reactions that occur in affected blood vessels and adjacent tissues in response to an injury or abnormal stimulation caused by a physical, chemical, or biologic agent. Inflammatory processes include: the local reactions and resulting morphologic changes; the destruction or removal of the injurious material; and the responses that lead to repair and healing. The typical signs of inflammation are redness, heat or warmth, swelling, pain, and occasionally inhibited or lost function. All of the signs may be observed in certain instances, although any particular sign is not necessarily always present. Inflammation often accompanies and is a response to infection or other injury, however, chronic and autoimmue inflammation represent undesirable pathological conditions in which infection is not typically present.

It is envisioned that methods of the present invention may be useful in the treatment of infection and inflammation. For example, galection-3-mediated increases in the migration of cells to the site of an infection or wound may accelerate the eradication of invading microorganisms of infection. Furthermore, galectin-3, galectin-3 binding polypeptides, and galectin-3 receptor binding polypeptides may facilitate localized migration to a desired therapeutic site while limiting migration of destructive cells to surrounding tissue, thereby decreasing tissue damage. In the inflammation phase, inflammatory cells, mostly neutrophils, enter the site of the wound followed by lymphocytes, monocytes, and later macrophages. The neutrophils that are stimulated begin to release proteases and reactive oxygen species (e.g., superoxide) into the surrounding medium with potential adverse effects on both the invading microorganisms and adjacent tissues. For example, the adhesion and spreading of activated neutrophils and monocytes to vascular endothelial cells with the subsequent release of toxio-oxidative metabolites and proteases has been implicated in the organ damage observed in diseases, such as, adult respiratory distress syndrome (ARDS; shock lung syndrome), glomerulonephritis, and inflammatory injury occurring after reperfusion of ischemic tissue such as to the heart, bowel, and central nervous system. (see, e.g., Harlan, Blood, 65: 513-525 (1985)).

Accordingly, methods for increasing migration of monocytes, neutrophils or macrophages to an inflammatory or infection site are provided comprising contacting the inflammatory or infection site, respectively with a migration-increasing amount of galectin-3, galectin-3 binding polypeptide or galectin-3 receptor binding polypeptide.

Methods are also provided for increasing migration of monocytes, neutrophils or macrophages to a tumor comprising contacting the tumor with a migration-increasing amount of galectin-3, galectin-3 binding polypeptide, or galectin-3 receptor binding polypeptide. “Tumor,” according to the present invention is any abnormal mass of tissue that results from excessive cell division that is uncontrolled and progressive. Tumors are also referred to as neoplasms. Tumors perform no useful body function. They may be either benign (not cancerous) or malignant and include localized as well as metastatic growths which may spread to locations distant to the site of the original tumor cell. It has been postulated that the basis for neoplastic development lies in the ability of an initial tumor cell to evade immune surveillance mechanism. Methods of enhancing immune surveillance of tumor cells, such as increasing monocyte, neutrophil or macrophage migration to a tumor, either alone or in combination with other therapy, may therefore prove useful in treating neoplastic diseases such as cancer.

The present invention also provides a method for indentifying an agent that modulates galectin-3 mediated cell migration comprising: contacting galectin-3 with a test agent; and detecting galectin-3 mediated cell migration, wherein an alteration of galectin-3 meditated cell migration in the presence of the test agent identifies an agent that modulates galectin-3 mediated cell migration. Agents according to the method may either increase or decrease galectin-3 mediated cell migration. In one embodiment, the agent is a small molecule, which may be naturally occurring or synthetic. In other embodiments, the agent may for example, be a co-factor, vitamin, hormone, enzyme, accelerant, stimulant, agonist, mimetic, antagonist, inhibitor, analog, ligand, or derivative. Also included are naturally occurring and synthetic biologicals, including proteins, peptides, polypeptides, lipids, carbohydrates, polysaccharides and sugars.

According to the method, galectin-3 may be contacted in vitro, such as in a test tube or other suitable vessel prior to or concurrent with detecting galectin-3 mediated migration. In one galectin-3 is contacted in vitro utilizing a micro Boyden chamber as described below. Contact may also occur intracellularly. Non-limiting examples of contacting galectin-3 intracellularly includes contacting intracellular or newly-synthesized forms of galectin-3 with agents capable of entering the cell, such as by diffusion or by active transport. Agents, including genes encoding biologicals such as polypeptides, may also be physically introduced into cells by such techniques as microinjection, electroporation, or transfection. Galectin-3 may also be contacted in vivo, such as by administering a systemic or local dose of an agent to an experimental animal. The agent may be administered by any route that places the agent in contact with galectin-3 in the animal. The dose may, for example, be administered subcutaneously (as described in Example 8 below) or intraperitoneally (as described below in Example 9).

The agent may interact directly or indirectly with galectin-3 to increase or decrease the effectiveness of galectin-3 in mediating cell migration. Also contemplated by the invention are agents that interact with galectin-3 receptors or other cellular structures. Such agents may, for example, block galectin-3 binding, thereby reducing cell migration mediated by either endogenous or exogenous galectin-3 in an organism. Conversely, agents that interact with galectin-3 receptors may act as agonists, thereby increasing galectin-3 mediated cell migration. Agents that act upon other components in galectin-3-mediated signal transduction pathways are non-limiting examples of additional agents contemplated by the invention.

Also provided by the present invention is an antibody that specifically binds galectin-3. One embodiment of the invention provides compositions containing migration-modulating amount galectin-3 antibodies and a pharmaceutically acceptable carrier, excipient or diluent.

Compositions comprising galectin-3 or a functional subsequence thereof and a pharmaceutically acceptable carrier, excipient or diluent are also included in the invention. “Functional subsequence” refers to any fragment or portion of galectin-3 possessing the desired experimental, clinical or therapeutic property of the intact galectin-3 molecule. Subsequences may be prepared by any means known in the art, such as by proteolytic digestion of intact, full-length galectin-3, by cloning and expressing fragments of a galectin-3 gene, or by synthesis of peptides by known chemical techniques.

In one aspect of this embodiment, compositions containing galectin-3 also contain a drug. The drug may include any compound, composition, biological or the like that potentiates, stabilizes or synergizes with galectin-3. Also included are drugs that may be beneficially or conveniently provided at the same time as galectin-3, such as drugs used to treat the same, a concurrent or a related symptom, condition or disease. In preferred embodiments, the drug may include without limitation anti-tumor, antiviral, antibacterial, anti-mycobacterial, anti-fungal, anti-cell proliferative or apoptotic agent. Drugs that are included in the compositions of the invention are well known in the art (see e.g., Goodman & Gilman's The Pharmacological Basis of Therapeutics, 9^{th} Ed. (Hardman, et al., eds) McGraw-Hill (1996) herein incorporated by reference).

Compositions of the present invention may be administered according to dosage regimens established in the art whenever specific pharmacological modification of galectin-3-mediated cell migration is desirable.

The present invention also provides pharmaceutical compositions comprising one or more compounds of the invention together with a pharmaceutically acceptable diluent, excipient, or carrier. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

lasses of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism. Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Antifungals include agents such as benzoic acid, undecylenic alkanolamide, ciclopiroxolamine, polyenes, imidazoles, allylamine, thicarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, ketoconazole, undecylenate, silver sulfadiazine, undecylenic acid, undecylenic alkanolamide and Carbol-Fuchsin.

Preferably such compositions are in unit dosage forms such as tablets, pills, capsules (including sustained-release or delayed-release formulations), powders, granules, elixirs, tinctures, syrups and emulsions, sterile parenteral solutions or suspensions, aerosol or liquid sprays, drops, ampoules, auto-injector devices or suppositories; for oral, parenteral (e.g. intravenous, intramuscular or subcutaneous), intranasal, sublingual or rectal administration, or for administration by inhalation or insufflation, and may be formulated in an appropriate manner and in accordance with accepted practices such as those disclosed in Remington's Pharmaceutical Sciences, (Gennaro, ed., Mack Publishing Co., Easton Pa., 1990, herein incorporated by reference). Alternatively, the compositions may be in sustained-release form suitable for once-weekly or once-monthly administration; for example, an insoluble salt of the active compound, such as the decanoate salt, may be adapted to provide a depot preparation for intramuscular injection. The present invention also contemplates providing suitable topical formulations for administration to, e.g. eye, skin or mucosa.

For instance, for oral administration in the form of a tablet or capsule, the active pharmacological drug component can be combined with an oral, non-toxic pharmaceutically acceptable inert carrier such as ethanol, glycerol, water and the like. Moreover, when desired or necessary, suitable binders, lubricants, disintegrating agents, flavoring agents and coloring agents can also be incorporated into the mixture. Suitable binders include, without limitation, starch, gelatin, natural sugars such as glucose, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For preparing solid compositions such as tablets, the active ingredient is mixed with a suitable pharmaceutical excipient, e.g. such as the ones described above, and other pharmaceutical diluents, e.g. water, to form a solid preformulation composition containing a homogeneous mixture of a compound of the present invention, or a pharmaceutically acceptable salt thereof. By the term “homogeneous” is meant that the active ingredient is dispersed evenly throughout the composition so that the composition may be readily subdivided into equally effective unit dosage forms such as tablets, pills and capsules. The solid preformulation composition may then be subdivided into unit dosage forms of the type described above containing from 0.001 to about 500 mg of the active ingredient of the present invention. The tablets or pills of the present composition may be coated or otherwise compounded to provide a dosage form affording the advantage of prolonged action. For example, the tablet or pill can comprise an inner core containing the active compound and an outer layer as a coating surrounding the core. The outer coating may be an enteric layer that serves to resist disintegration in the stomach and permits the inner core to pass intact into the duodenum or to be delayed in release. A variety of materials can be used for such enteric layers or coatings, such materials including a number of polymeric acids and mixtures of polymeric acids with conventional materials such as shellac, cetyl alcohol and cellulose acetate.

The liquid forms in which the present compositions may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical carriers. Suitable dispersing or suspending agents for aqueous suspensions include synthetic and natural gums such as tragacanth, acacia, alginate, dextran, sodium carboxymethylcellulose, gelatin, methylcellulose or polyvinylpyrrolidone. Other dispersing agents that may be employed include glycerin and the like. For parenteral administration, sterile suspensions and solutions are desired. Isotonic preparations, which generally contain suitable preservatives, are employed when intravenous administration is desired. The compositions can also be formulated as an ophthalmic solution or suspension formation, i.e., eye drops or ointment, for ocular administration Consequently, the present invention also relates to a method of alleviating or treating a disease, symptom or condition in an animal in which galectin-3-mediated modulation of cell migration, in particular modulation of monocytes, macrophages, and/or neutrophils, has a beneficial effect, by administering a therapeutically effective amount of a galectin-3, a functional subsequence thereof, a galectin-3 binding polypeptide or a galectin-3 receptor binding polypeptide, such as an antibody or other compositions of the present invention to a subject in need of such treatment. Such diseases or conditions may, for instance arise from inappropriate, undesirable or inadequate migration of monocytes, macrophages, and/or neutrophils, such as encountered in inflammation, infection, and neoplasia.

In the methods of the invention in which a detectable result or beneficial effect is a desired outcome, such as a therapeutic benefit in a subject treated in accordance with the invention, compositions such as binding agents can be administered in sufficient or effective amounts. The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes palliation or alleviation of any of the symptoms of the disease being treated. Particularly, therapeutically effective amounts of the compositions of the present invention may be useful for treating the symptoms of inflammation, infection and neoplasia.

As used herein, an “amount sufficient” or “amount effective” refers to an amount of a composition (e.g., a galectin-3 inhibitor) that provides, in single or multiple doses, alone or in combination with one or more other (second) compounds or agents (e.g., a drug), treatments or therapeutic regimens, a long or short term detectable response, a desired outcome or beneficial effect in a given subject of any measurable or detectable degree or duration (e.g., for minutes, hours, days, months, years, or cured).

An amount sufficient or an amount effective can but need not be provided in a single administration and can but need not be administered alone (i.e., without a second drug, agent, treatment or therapeutic regimen), or in combination with another compound, agent, treatment or therapeutic regimen. In addition, an amount sufficient or an amount effective need not be sufficient or effective if given in single or multiple doses without a second compound, agent, treatment or therapeutic regimen, since additional doses, amounts or duration above and beyond such doses, or additional drugs, agents, treatment or therapeutic regimens may be included in order to be effective or sufficient in a given subject. Further, an amount sufficient or an amount effective need not be effective in each and every subject, nor a majority of subjects in a given group or population. Thus, an amount sufficient or an amount effective means sufficiency or effectiveness in a particular subject, not a group or the general population. As is typical for such methods, some subjects will exhibit a greater or less response to a method of the invention, including treatment/therapy.

An “amount sufficient” or “amount effective” includes reducing, preventing, delaying or inhibiting onset, reducing, inhibiting, delaying, preventing or halting the progression or worsening of, reducing, relieving, alleviating the severity, frequency, duration, susceptibility or probability of one or more adverse or undesirable symptoms associated with the condition, disorder or disease of the subject. In addition, hastening a subject's recovery from one or more adverse or undesirable symptoms associated with the condition, disorder or disease is considered to be an amount sufficient or effective. Various beneficial effects and indicia of therapeutic benefit are as set forth herein and are known to the skilled artisan.

An “amount sufficient” or “amount effective,” in the appropriate context, can refer to therapeutic or prophylactic amounts. Therapeutically or prophylactically sufficient or effective amounts mean an amount that detectably improves the condition, disorder or disease, such as asthma or, respiratory airway or mucosal disorder, as assessed by one or more objective or subjective clinical endpoints appropriate for the condition, disorder or disease.

Advantageously, compositions of the present invention may be administered one, two, three, four, five, or more times daily, weekly, monthly or annually. Total daily dosage may be administered in divided doses two, three, four or more times daily. The compositions administered to the subject can be administered concurrently with, or within about 1-60 minutes, hours, or days of the onset of a symptom of a disorder or associated with a condition (e.g., allergic asthma, an asthmatic episode or airway-constriction or obstruction). Furthermore, compounds for the present invention may be administered in intranasal form via topical use of suitable intranasal vehicles, or via transdermal routes, using those forms of transdermal skin patches well known to persons skilled in the art. To be administered in the form of a transdermal delivery system, the dosage administration will, of course, be continuous rather than intermittent throughout the dosage regimen. annually.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound employed. A physician or veterinarian of ordinary skill can readily determine and prescribe the effective amount of the drug required to prevent, counter the progress of, or arrest or alleviate the symptoms of the disease or disorder that is being treated.

The daily dosage of the products may be varied over a wide range, such as from 0.001 to 100 mg per adult human per day. The amount administered can be about 0.00001 mg/kg, to about 10,000 mg/kg, about 0.0001 mg/kg, to about 1000 mg/kg, about 0.001 mg/kg, to about 100 mg/kg, about 0.01 mg/kg, to about 10 mg/kg, about 0.1 mg/kg, to about 1 mg/kg one, two, three, four, or more times per hour, day, week, month or more. For oral administration, the compositions can be provided in the form of tablets containing 0.001, 0.01, 0.05, 0.1, 0.5, 1.0, 2.5, 5.0, 10.0, 15.0, 25.0, 50.0, 100.0, 250.0, or 500.0 mg of the active ingredient for the symptomatic adjustment of the dosage to the patient to be treated.

A unit dose typically contains from about 0.001 mg to about 500 mg of the active ingredient, preferably from about 0.1 mg to about 100 mg of active ingredient, more preferably from about 1.0 mg to about 10 mg of active ingredient. An effective amount of the drug is ordinarily supplied at a dosage level of from about 0.0001 mg/kg to about 25 mg/kg of body weight per day. Preferably, the range is from about 0.001 to 10 mg/kg of body weight per day, and especially from about 0.001 mg/kg to 1 mg/kg of body weight per day. The compounds may be administered on a regimen of, for example, 1 to 4 or more times per day.

Compositions according to the present invention may be used alone at appropriate dosages defined by routine testing in order to obtain optimal pharmacological effect on cell migration, in particular monocyte, macrophage, and/or neutrophil migration, while minimizing any potential toxic or otherwise unwanted effects. In addition, co-administration or sequential administration of other agents or drugs, which improve the effect of the compositions of the invention may, in some cases, be desirable. For example, it may be desirable to administer galectin-3 or a functional subsequence thereof together with anti-tumor, antiviral, antibacterial, anti-mycobacterial, anti-fungal, anti-cell proliferative or apoptotic agent.

According to the present invention, compositions comprising galectin-3 or a functional subsequence thereof and an article of manufacture are also included. In one embodiment, the article of manufacture comprises a dressing. Preferably, the dressing is a bandage, suture, sponge, or a surgical dressing. Bandages, sutures, sponges or surgical dressings may be made of any suitable material known in the art, such as cotton gauze, adhesive tapes (including paper), latex, Dacron, Gortex, nylon, Prolene, Vicryl and gut. In one aspect, the article of manufacture facilitates delivery of the galectin-3, subsequence, or another composition, such as a drug. In another aspect, the article of manufacture provides a related function, such as promoting wound healing, maintaining sterility of a surgical site or facilitating drainage.

The compositions of the invention may advantageously be administered in a depot or sustained release form. Alternatively, administration may be by continuous or intermittent infusion, injection, insufflation or infiltration. The invention therefore includes a microfabricated device containing galectin-3 or a functional subsequence thereof in a pharmaceutically acceptable carrier, the device capable of controlled delivery of the galectin-3 or the functional subsequence. “Microfabricated device” refers to a structure having chambers and at least way-one flow, generally accommodating small volumes; for example, chambers generally accommodate volumes that range from about 0.01 μl to about 10 ml. In one embodiment, the device includes an internal or external pump. In a preferred embodiment, the device can be implanted in the body of a subject. In various aspects the device may be implanted at the site of infection, in close proximity to or within a solid tumor or at the site of a lesion.

The invention further provides methods for treating asthma. In one embodiment, a method includes administering to a subject having or at risk of having an acute or chronic asthmatic episode or an asthma associated symptom, an inhibitor of galectin-3 expression or activity in an amount sufficient to treat asthma.

The invention also provides methods for reducing or decreasing onset, progression, severity, frequency, duration or probability of one or more symptoms associated with asthma (e.g., one or more adverse physiological or psychological symptoms associated with allergic asthma). In one embodiment, a method includes administering to a subject an amount of inhibitor of galectin-3 expression or activity sufficient to reduce or decrease onset, progression, severity, frequency, duration or probability of the one or more symptoms associated with asthma.

Exemplary classes and non-limiting particular examples of inhibitors of galectin-3 expression or activity useful in accordance with the invention methods are as set forth herein or are known in the art. Exemplary asthma symptoms can be caused by an allergen or by exercise. Specific non-limiting examples of asthma symptoms include lung, airway or respiratory mucosal inflammation or tissue damage, shortness of breath, wheezing, coughing, chest-tightness, chest pain, increased heart rate, runny nose, airway-constriction or obstruction, decreased lung capacity, and an acute asthmatic episode. Asthma associated symptoms can be chronic or acute, such as a chronic or acute asthmatic episode. Invention methods are further applicable to treatment of bronchial asthma; allergic rhinitis; allergic conjunctivitis and eosinophilia.

The invention moreover provides methods for treating a respiratory disorder or a respiratory airway or respiratory mucosal disorder. In one embodiment, a method includes administering to a subject having or at risk of having an acute or chronic a respiratory disorder or a respiratory airway or respiratory mucosal disorder or an associated symptom, an inhibitor of galectin-3 expression or activity in an amount sufficient to treat the respiratory disorder or the respiratory airway or respiratory mucosal disorder. Methods of the invention include reducing, decreasing, inhibits, delaying, eliminating or preventing onset, probability, severity, frequency, or duration of one or more symptoms associated with or caused by the respiratory disorder or the respiratory airway or respiratory mucosal disorder. Exemplary respiratory airway disorders include allergic airway inflammation. Additional non-limiting examples of respiratory airway and respiratory mucosal disorders include: Airway Obstruction, Apnea, Asbestosis, Atelectasis, Berylliosis, Bronchiectasis, Bronchiolitis, Bronchiolitis Obliterans Organizing Pneumonia, Bronchitis, Bronchopulmonary Dysplasia, Cough, Empyema, Pleural Empyema, Pleural Epiglottitis, Hemoptysis, Kartagener Syndrome, Meconium Aspiration, Pleural Effusion, Pleurisy, Pneumonia, Pneumothorax, Respiratory Distress Syndrome, Respiratory Hypersensitivity, Respiratory Tract Infections, Rhinoscleroma, Scimitar Syndrome, Severe Acute Respiratory Syndrome, Silicosis, Tracheal Stenosis and Whooping Cough.

The invention additionally provides methods for reducing or decreasing the probability, severity, frequency, duration or preventing a subject from having an acute asthmatic episode (e.g., caused by allergic asthma). In one embodiment, a method includes administering to a subject that has previously experienced an asthmatic episode or has been diagnosed as having asthma with an amount of an inhibitor of galectin-3 expression or activity sufficient to reduce or decrease onset, probability, severity, frequency, duration or prevent an acute asthmatic episode.

Methods of the invention also include inducing or increasing airway-dilation, as well as methods of decreasing probability, severity, frequency, duration or preventing airway-constriction or obstruction. In one embodiment, a method includes administering to a subject in need of increased airway-dilation an amount of an inhibitor of galectin-3 expression or activity sufficient to induce or increase airway-dilation in the subject. In another embodiment, a method includes administering to a subject in need of reducing the probability, severity, frequency, duration or preventing airway-constriction or obstruction an amount of an inhibitor of galectin-3 expression or activity sufficient to reduce or decrease the probability, severity, frequency, duration or prevent airway-constriction or obstruction in the subject.

Methods of the invention can include contacting or administering a second agent (e.g., drug) to the subject prior to, concurrently with or following contacting or administering an inhibitor of galectin-3 expression or activity. In various aspects, a second agent (e.g., drug) is an anti-inflammatory, anti-asthmatic or anti-allergy drug, a hormone or a steroid. In various additional aspects, a second agent (e.g., drug) is an anti-histamine, anti-leukotriene (e.g., cysteinyl-leukotriene (Cys-LT)), anti-IgE, anti-α4 integrin, anti-β2 integrin, anti-CCR3 antagonist, β2 agonist (e.g., β2-adrenoceptor) or anti-selectin or glucocorticoid.

The term “subject” includes animals, typically mammalian animals, such as but not limited to humans, non-human primates (apes, gibbons, chimpanzees, orangutans, macaques), domestic animals (dogs and cats), farm animals (horses, cows, goats, sheep, pigs), and experimental animals (mouse, rat, rabbit, guinea pig). Subjects include animal disease models (e.g., asthma, allergy). Subjects include naturally occurring or non-naturally occurring mutated or non-human genetically engineered (e.g., transgenic or knockout) animals.

Subjects having or at risk of having a condition, disorder or disease treatable in accordance with the invention methods include subjects with an existing condition or a known or a suspected predisposition towards developing a the condition or a symptom associated with the conditions, disorders and diseases set forth herein. Subjects further include animals having or at risk of having a chronic or acute condition, disorder or disease. At risk subjects include those at risk or predisposed towards suffering from such conditions, disorders or diseases based upon their prior or a family history, but the condition, disorder or disease may not or only mildy manifests itself in the subject. At risk subjects can be identified by a personal or family history, through genetic screening, tests appropriate for detection of increased risk, or exhibiting relevant symptoms indicating predisposition or susceptibility.

Subjects in need of treatment in accordance with the invention include subjects having or at risk of having asthma (diagnosed as or at risk of having acute or chronic asthma), respiratory airway or mucosal disorder, airway constriction or obstruction, for example. A “subject having or at risk of having asthma” refers to a subject suffering from an acute episode of asthma, either a new-onset or a recurrent episode, a subject with a prior history of one or more episodes of asthma, or a subject with a known or suspected predisposition towards developing asthma. A subject having asthma can have active asthma or can be asymptomatic and between acute asthma episodes. A subject having asthma can be suffering from recently acute asthmatic episode (e.g., within minutes or hours of episode onset). A subject having asthma can have a positive skin test, or exhibit one or more symptoms typically associated with acute or chronic asthma, for example, a symptom of allergic asthma. A subject having or at risk of having asthma may be or has been exposed to an allergen, for example, and is at increased risk of suffering from an asthmatic episode due to a predisposition or susceptibility towards an asthmatic episode upon re-exposure to the allergen. Subjects predisposed or susceptible to, exposed to or allergic to these or other allergens are at risk of having asthma and, therefore, are amenable to treatment in accordance with the invention.

At risk subjects also appropriate for treatment in accordance with the invention include subjects exposed to an allergen or are susceptible to having an allergic reaction, or infection or exposure by an agent that is associated with an allergy or allergic reaction. At risk subjects appropriate for treatment in accordance with the invention include subjects having a predisposition towards an allergic reaction, or infection or exposure to an agent that is associated with an allergy or allergic reaction due to a genetic or environmental risk factor. Methods of the invention include subjects contacted with or administered to a binding agent prophylactically.

Treatment can provide a beneficial effect, such as reducing, inhibiting, decreasing, delaying, halting, eliminating or preventing progression, severity, frequency, duration, susceptibility or probability of developing one or more symptoms associated with the asthma (acute or chronic), respiratory airway or mucosal disorder, airway constriction or obstruction.

The term “associated,” when used in reference to the relationship between a symptom and a condition, disorder or disease, means that the symptom is caused by the condition, disorder or disease, or is a secondary effect of the condition, disorder or disease. A symptom that is present in a subject may therefore be the direct result of or caused by the condition, or may be due at least in part to the subject reacting or responding to the condition, disorder or disease. For example, symptoms that occur during an allergic episode are due in part to hypersensitivity or an aberrant response of the immune system of the subject to the allergen.

“Asthma” refers to an allergic or non-allergic condition, disorder or disease of the respiratory system that is episodic and characterized by inflammation with constriction, narrowing or obstruction of the airways. Allergic asthma is typically associated with increased reactivity of respiratory system (airways, lung, etc.) to an inhaled agent. Asthma is frequently, although not exclusively associated with atopic or allergic symptoms. Typically, a subject with asthma suffers from recurrent attacks of paroxysmal dyspnea (i.e., “reversible obstructive airway passage disease”), cough, shortness of breath with wheezing due to spasmodic contraction of the bronchi, sometimes referred to as “bronchospasm,” chest pain, chest tightness, etc. While a plurality of such adverse symptoms typically occur in asthma, the existence of any one is usually adequate for diagnosis of asthma, and for treatment in accordance with the invention.

Asthmatic conditions include allergic asthma as well as bronchial allergy, which typically are provoked by a variety of factors including exercise such as vigorous exercise (“exercise-induced bronchospasm”), and irritant particles (allergens such as pollen, dust, venoms, cotton, dander, foods). Asthmatic conditions can be acute, chronic, mild, moderate or severe asthma (unstable asthma), nocturnal asthma or asthma associated with psychologic stress.

“Allergic rhinitis” is an allergic reaction of the nasal mucosa (upper airways), which includes hay fever (seasonal allergic rhinitis) and perennial rhinitis (non-seasonal allergic rhinitis) which are typically characterized by seasonal or perennial sneezing, rhinorrhea, nasal congestion, pruritis and eye itching, redness and tearing. “Non-allergic rhinitis” refers to eosinophilic non-allergic rhinitis, in subjects with negative skin tests, and subjects who have abnormal or undesirable numbers of eosinophils in their nasal secretions.

A “respiratory airway disorder” or a “respiratory mucosal disorder” means a condition, disorder or disease related to a tissue or organ of the respiratory system. Examples include, but are not limited to, upper or lower airway inflammation, allergy(ies), breathing difficulty, cystic fibrosis (CF), allergic rhinitis (AR), Acute Respiratory Distress Syndrome (ARDS), pulmonary hypertension, lung inflammation, bronchitis, airway obstruction, airway constriction, airway narrowing, broncho-constriction and inflammation associated with microbial or viral infections, such as picornaviridae (rhinoviruses such as human rhinovirus (HRV); enteroviruses (EV) such as polioviruses, coxsackieviruses and echoviruses; and hepatitis A virus) or severe acute respiratory syndrome (SARS). Additional non-limiting examples of respiratory airway disorders and respiratory mucosal disorders include apnea, asbestosis, atelectasis, berylliosis, bronchiectasis, bronchiolitis, bronchiolitis obliterans Organizing Pneumonia, Bronchitis, Bronchopulmonary Dysplasia, Common Cold, Cough, Empyema, Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Respiratory Tract Infections, Rhinoscleroma, Scimitar Syndrome, Severe Acute Respiratory Syndrome, Silicosis, Tracheal Stenosis and Whooping Cough.

The term “airway,” as used herein, means a part of or the whole respiratory system of a subject that is exposed to air. “Airways” therefore include the upper and lower airway passages, within which are not limited to the trachea, bronchi, bronchioles, terminal and respiratory bronchioles, alveolar ducts and alveolar sacs. Airways include sinuses, nasal passages, nasal mucosum and nasal epithelium. The airway also includes, but is not limited to throat, larynx, tracheobronchial tree and tonsils.

Reducing, inhibiting decreasing, eliminating, delaying, halting or preventing a progression or worsening or an adverse symptom of the condition, disorder or disease is a satisfactory outcome. The dose amount, frequency or duration may be proportionally increased or reduced, as indicated by the status of the condition, disorder or disease being treated, or any adverse side effects of the treatment or therapy. Dose amounts, frequencies or duration also considered sufficient and effective are those that result in a reduction of the use of another drug, agent, treatment or therapeutic regimen or protocol. For example, a galectin-3 inhibitor is considered as having a beneficial or therapeutic effect if contact, administration or delivery in vivo results in the use of a lesser amount, frequency or duration of another drug, agent, treatment or therapeutic regimen or protocol to treat the condition, disorder or disease, or an adverse symptom thereof.

In accordance with the invention, there are provided methods which provide a beneficial effect, such as a therapeutic benefit, to a subject. In one embodiment, a method reduces the probability, susceptibility, severity, frequency, duration or prevents an acute or chronic asthmatic episode (e.g., associated with allergic or non-allergic asthma) in a subject. In another embodiment, a method increases, stimulates, enhances, induces or promotes airway-dilation in the subject. In an additional aspect, a method reduces the probability, susceptibility, severity, frequency, duration or prevents or eliminates airway-constriction or obstruction in the subject. In a further aspect, a method is sufficient to reduce progression, severity, frequency, duration, susceptibility, probability, halt, eliminate or prevent one or more adverse physiological or psychological symptoms associated with asthma (allergic or non-allergic).

Sufficiency or effectiveness of a particular treatment can be ascertained by various clinical indicia and endpoints. For example, in order to ascertain an improvement in asthma, an increase in airway dilation, lung function or a reduction in airway constriction, obstruction or narrowing, progression, severity, duration, frequency, susceptibility or probability of one or more symptoms of asthma. A “therapeutically effective” or an “amount sufficient” or “amount effective” to treat asthma is therefore an amount that provides an objective or subjective reduction or improvement in progression, severity, frequency, susceptibility or probability of lung or airway inflammation, lung or airway tissue damage, shortness of breath, wheezing, coughing, chest-tightness, chest pain, increased heart rate, runny nose, airway or broncho-constriction or -obstruction or narrowing, decreased lung capacity, acute asthmatic episodes and nighttime awakenings. Thus, a reduction, decrease, inhibition, delay, halt, prevention or elimination of one or more adverse symptoms (e.g., shortness of breath, wheezing, coughing, chest-tightness, chest pain, increased heart rate, runny nose, acute asthmatic episodes and nighttime awakenings) can be used as a measure of sufficiency or effectiveness.

A method to determine an improvement in lung or pulmonary function is to measure the forced expiratory volume in one second (FEV₁) an increase of which indicates an improvement. Spirometry is a test which measures the amount and rate at which air can pass through airways. Airway narrowing due to inflammation restricts air flow through the airways, which is detected by changed spirometry values. Exercise challenge and methacholine inhalation tests are also used to evaluate airway narrowing or constriction. Yet another method to determine an improvement is to measure serum IgE in a subject. A reduction in serum or bronchoalveolar lavage (BAL) fluid IgE is an objective measure of treatment efficacy. Various additional methods are known in the art for detecting improvement in lung or pulmonary function.

The terms “treat,” “therapy” and grammatical variations thereof when used in reference to a method means the method provides an objective or subjective (perceived) improvement in a subjects' condition, disorder or disease, or an adverse symptom associated with the condition, disorder or disease. Non-limiting examples of an improvement can therefore reduce or decrease the probability, susceptibility or likelihood that the subject so treated will manifest one or more symptoms of the condition, disorder or disease. Additional symptoms and physiological or psychological responses caused by or associated with conditions, disorders or diseases associated with, for example, asthma are set forth herein and known in the art and, therefore, improvements in these and other adverse symptoms or physiological or psychological responses can also be included in the methods of the invention.

Methods of the invention therefore include providing a detectable or measurable beneficial effect or therapeutic benefit to a subject, or any objective or subjective transient or temporary, or longer-term improvement (e.g., cure) in the condition. Thus, a satisfactory clinical endpoint is achieved when there is an incremental improvement in the subjects condition or a partial reduction in the severity, frequency, duration or progression of one or more associated adverse symptoms or complications or inhibition, reduction, elimination, prevention or reversal of one or more of the physiological, biochemical or cellular manifestations or characteristics of the condition, disorder or disease. A therapeutic benefit or improvement (“ameliorate” is used synonymously) therefore need not be complete ablation of any or all adverse symptoms or complications associated with the condition, disorder or disease but is any measurable or detectable objectively or subjectively meaningful improvement in the condition, disorder or disease. For example, inhibiting a worsening or progression of the condition, disorder or disease, or an associated symptom (e.g., slowing or stabilizing one or more symptoms, complications or physiological or psychological effects or responses), even if only for a few days, weeks or months, even if complete ablation of the condition, disorder or disease, or an associated adverse symptom is not achieved is considered to be beneficial effect.

Prophylactic methods are included. “Prophylaxis” and grammatical variations thereof mean a method in accordance with the invention in which contact, administration or in vivo delivery to a subject is prior to manifestation or onset of a condition, disorder or disease (or an associated symptom or physiological or psychological response), such that it can eliminate, prevent, inhibit, decrease or reduce the probability, susceptibility or frequency of having a condition, disorder or disease, or an associated symptom. Target subject's for prophylaxis can be one of increased risk (probability or susceptibility) of contracting the condition, disorder or disease, or an associated symptom, or recurrence of a previously diagnosed condition, disorder or disease, or an associated symptom, as set forth herein and known in the art.

Any compound or agent (e.g., drug), therapy or treatment having a beneficial, additive, synergistic or complementary activity or effect (beneficial or therapeutic) can be used in combination with a binding agent in accordance with the invention. Methods of the invention therefore include combination therapies and treatments.

Pharmaceutical compositions can optionally be formulated to be compatible with a particular route of administration. Thus, pharmaceutical compositions include carriers (excipients, diluents, vehicles or filling agents) suitable for administration by various routes and delivery to targets, locally, regionally or systemically.

Exemplary routes of administration for contact or in vivo delivery which a composition can optionally be formulated include respiratory system (nasal, inhalation, respiration, intubation, intrapulmonary instillation), oral, buccal, intrapulmonary, rectal, intrauterine, intradermal, topical, dermal, parenteral, sublingual, subcutaneous, intravascular, intrathecal, intraarticular, intracavity, transdermal, iontophoretic, intraocular, ophthalmic, optical, intravenous, intramuscular, intraglandular, intraorgan, intralymphatic.

Nasal and instillation formulations typically include aqueous solutions of active ingredient (compounds or agents) optionally with one or more preservative or isotonic agents. Such formulations are typically adjusted to a pH and isotonic state compatible with nasal mucous membranes. A solvent may include only water, or it may be a mixture of water and one or more other components (e.g., ethanol).

Formulations that include respirable or inhalable liquid or solid particles of the active ingredient (e.g., compound, binding agent) can have particles of a size sufficiently small to pass through the mouth and larynx upon inhalation and continue into the airways of the lungs (e.g., bronchi and alveoli). Particles typically range in size from about 0.05, about 0.1, about 0.5, about 1, about 2 to about 4, about 6, about 8, about 10 microns in diameter. Particles of non-respirable size can be included in an aerosol or spray to deposit in the throat. For nasal administration or intrapulmonary instillation, a particle size in the range of about 8, about 10, about 20, about 25 to about 35, about 50, about 100, about 150, about 250, about 500 μm (diameter) is typical for retention in nasal cavity or for instillation into lung.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof.

Galectin-3 inhibitors and pharmaceutical formulations can be administered into the respiratory system of a subject by inhalation, respiration, intubation, or intrapulmonary instillation (into the lungs), for example. Respiratory administration can be achieved using an aerosol or spray of a gas, liquid or powdered nasal, intrapulmonary, respirable or inhalable in a particle form. The particles include the compound or binding agent, and optionally any other component (e.g., second compound), and are administered or delivered to the subject by inhalation, by nasal administration or instillation into the airways or the lung.

Administration to airways can be accomplished using an article of manufacture, such as container with or without an aerosol. Liquid formulations may be squirted into the respiratory system (e.g., nose) and the lung from a container by pressure or using an aerosol propellant. or a spray device or delivery system. Administration can be passive or it can be assisted by a pressurized delivery system or device. An aerosol, delivery system or device can include a pressurized container containing liquid, gas or dry powder.

An “aerosol formulation” refers to a preparation that includes droplets or particles of active ingredient (e.g., compound, binding agent) suitable for delivery to respiratory system (e.g., lung, airway, nasal and sinus epithelium). The aerosol formulation can include a sufficient or effective amount of a compound or agent and a pharmaceutically acceptable carrier, optionally a propellant, in a container or aerosol or spray device or delivery system. Aerosol formulations can deliver high concentrations into airways with relatively low systemic absorption, and include for example nasal sprays, inhalation solutions, inhalation suspensions, and inhalation sprays. Nasal sprays typically contain active ingredient dissolved or suspended in solution or in an excipient, in nonpressurized dispensers that deliver a metered dose of the ingredient.

For aerosol delivery, pH of the formulation is typically between 5.0 and 7.0. If the aerosol is too acidic or basic, it can cause bronchospasm and cough. The tolerized pH range is relative and depends on a patient's tolerance: some patients tolerate a mildly acidic aerosol, which in others will cause bronchospasm. Typically, an aerosol formulation having a pH less than 4.5 induces bronchospasm.

Compositions including compounds and binding agents can be formulated in a dry powder for delivery into the endobronchial space. Dry powder formulations provide stability, high volume delivery per puff, and low susceptibility to microbial growth. Dry powder formulations typically are stable at ambient temperature, and have a physiologically acceptable pH of 4.0-7.5. Dry powder formulations can be used directly in metered dose or dry powder inhalers.

Aerosol and spray delivery systems and devices, also referred to as “aerosol generators” and “spray generators” are known in the art and include metered dose inhalers (MDI), nebulizers (ultrasonic, electronic and other nebulizers), nasal sprayers and dry powder inhalers.

MDIs typically include an actuator, a metering valve, and a container that holds a suspension or solution, propellant, and surfactant (e.g., oleic acid, sorbitan trioleate, lecithin). The container may be pressurized or not, but typically it is either squeezed to dispense the ingredient, or has an actuator connected to a metering valve so that activation of the actuator causes a predetermined amount to be dispensed from the container in the form of an aerosol, which is inhaled by the subject. MDIs typically use liquid propellant. Typically, metered-dose aerosol inhalers create droplets that are 15 to 30 microns in diameter. Currently, MDI technology is optimized to deliver masses of 1 microgram to 10 mg of a therapeutic.

Nebulizers, also referred to as atomizers, are devices that turn medication into a fine mist inhalable by a subject through a face mask that covers the mouth and nose. Nebulizers provide small droplets and high mass output which can be delivered to upper and lower respiratory airways. Typically, nebulizers create droplets down to about 1 micron in diameter. Doses administered by nebulizers are typically larger than doses administered by MDIs.

Nebulizers include air-jet and ultrasonic nebulizers, in fluid connection with a reservoir containing disposed therein a solution or suspension of active ingredient. Nebulizers (air-jet, ultrasonic or electronic) are typically used for acute care of nonambulatory patients and in infants and children. Airjet nebulizers are relatively large but considered portable because of the availability of small compressed air pumps. Ultrasonic and electronic nebulizers are typically more portable because they usually do not require a source of compressed air. An example of an airjet nebulizer is the NE-C25 CompAir XLT Compressor Nebulizer System (Omron® Healthcare). Examples of ultrasonic nebulizers include the Zewa Portable Ultrasonic Nebulizer (Zewa, Inc.); the MabisMist II Ultrasonic Nebulizer (Mabis Healthcare, Inc.); and the MICROAir Ultrasonic Nebulizer (Omron® Healthcare). An example of an electronic nebulizer is the Micro-Air® Electronic Nebulizer with V.M.T. (Omron® Healthcare). Modified nebulizers can have the addition of a one-way flow valve (e.g., Pari LC Plus™, Pari Respiratory Equipment, Inc.), which delivers up to 20% more drug than unmodified nebulizers.

Components of the nebulizer are typically made of a material suitable for their intended function. The housing of the nebulizer and, if the function allows, other parts can be made of plastic (PVC, Polycarbonate, polystyrene, polypropylene, polybutylene, etc.). Plastic can be formed by injection molding. For medical applications, physiologically acceptable materials are used.

Dry-powder inhalers (DPI) can be used to deliver the compounds or agents, either alone or in combination with a pharmaceutically acceptable carrier, second compound, etc. Dry powder inhalers deliver active ingredient to airways and lungs while the subject inhales through the device. DPIs typically do not contain propellants or any other ingredients, only the medication, but may optionally include other components. DPIs are typically breath-activated, but may involve air or gas pressure to assist delivery. For breath-activated DPIs, a subject need not coordinate breathing with the activation of the inhaler.

An aerosol, delivery system or device can include a propellant. Exemplary propellants include chlorofluorocarbons (e.g., trichlorofluoromethane, dichlorodifluoromethane, dichlorotetrafluoromethane, CFC-11, CFC-12) and the non-d chlorofluorocarbons, HFC-134A and HFC-227. Suitable fluorocarbon (HFA) propellants are known in the art and include, for example, HFA 134a (1,1,1,2-tetrafluoroethane), HFA227 (1,1,1,2,3,3,3-heptafluoro-n-propane) and mixtures of HFA134a and HFA227.

Pharmaceutical compositions and delivery systems appropriate for compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

The invention provides kits including compositions (e.g., galectin-3 inhibitor) suitable for practicing the methods, treatment protocols or therapeutic regimes herein, and suitable packing material. In one embodiment, a kit includes a galectin-3 inhibitor, and instructions for administering said galectin-3 inhibitor to a subject (e.g. to lungs or airways of a subject).

The term “packing material” refers to a physical structure housing a component of the kit. The material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to a ampule, tube or vial containing a kit component. Labels or inserts can additionally include a computer readable medium, such as a disk (e.g., floppy diskette, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein (e.g., the binding agent or pharmaceutical composition), dose amounts, clinical pharmacology of the active agent(s) including mechanism of action, pharmacokinetics and pharmacodynamics. Labels or inserts can include information identifying manufacturer information, lot numbers, manufacture location and date.

Labels or inserts can include information on a condition, disorder or disease for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or therapeutic regimes described herein. Exemplary instructions include, instructions for performing a method of the invention as set forth herein or known in the art.

Labels or inserts can include information on any benefit that a component may provide, such as a therapeutic benefit. For example, a non-limiting example of a benefit would be improved breathing, increased airway dilation. A benefit could also include a reduced need (amount, frequency or duration) for other medications, treatment protocols or therapeutic regimes, that the subject may be using or have used for treatment of the condition, disorder or disease.

Labels or inserts can include information on potential adverse side effects, such as warnings to the subject or clinician regarding situations where it would not be appropriate to use a particular composition (e.g., a galectin-3 inhibitor). For example, adverse side effects are generally more likely to occur at higher dose amounts, frequency or duration of the active agent and, therefore, instructions could include recommendations against higher dose amounts, frequency or duration. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

A kit can contain include a components, such as a device suitable for practicing methods, treatment protocols or therapeutic regimes described herein. The device can be used to contact, administer or for in vivo delivery to a subject. The device can be a container, aerosol or spray generator, (e.g., MDI, nebulizer or DPI), vessel or holder for delivery of a compound or agent (e.g., a galectin-3 inhibitor) to a subject. A non-limiting example of such a device is metered-dose inhaler (MDI) for oral inhalation, which may be pressurized (see, for example U.S. Pat. No. 6,131,566). Suitable packaging for an MDI is described in WO 2000/37336 A1.

In one particular embodiment, a kit includes an inhibitor of galectin-3 expression or activity, and instructions for administering said inhibitor to a subject in an amount sufficient to treat asthma. In another particular embodiment, a kit includes an inhibitor of galectin-3 expression or activity, and instructions for administering said inhibitor to a subject in an amount sufficient to reduce or decrease onset, progression, severity, frequency, duration or probability of one or more symptoms associated with asthma. In a further particular embodiment, a kit includes an inhibitor of galectin-3 expression or activity, and instructions for administering said inhibitor to a subject in an amount sufficient to treat a respiratory disorder or a respiratory airway or respiratory mucosal disorder. In still another particular embodiment, a kit includes an inhibitor of galectin-3 expression or activity, and instructions for administering said inhibitor to a subject in an amount sufficient to treat a respiratory disorder or a respiratory airway or respiratory mucosal disorder. In still further particular embodiments, a kit includes an inhibitor of galectin-3 expression or activity, and instructions for administering said inhibitor to a subject in an amount sufficient to reduce or decrease the probability, severity, frequency, duration or prevent a subject from having an acute asthmatic episode; and instructions for administering said inhibitor to a subject in an amount sufficient to increase airway-dilation, or to reduce or decrease probability, severity, frequency, duration or prevent airway-constriction or obstruction.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “an inhibitor of galectin-3” includes a plurality of such inhibitors and reference to “a symptom” can include reference to one or more symptoms, and so forth.

A number of embodiments of the invention have been described. Nevertheless, it will be understood that various modifications may be made without departing from the spirit and scope of the invention. Accordingly, the following examples further illustrate the present invention, but should not be construed as in any way limiting its scope.

### EXAMPLES

A. Materials.

Recombinant human galectin-3 (Hsu, et al., J. Biol. Chem. 267:14167-74 (1992)) the C-terminal domain fragment of galectin-3 (galectin-3C) (Yang et al., Proc. Natl. Acad. Sci. USA 93:6736-42 (1996)), a mouse monoclonal antibody against galectin-3 (B2C10) (Liu, et al., Biochemistry 35:60773-79 (1996)), and mouse monoclonal anti-DNP IgG1 (Liu, et al., J. Immunol. 124:2728-31 (1980)) were prepared as described previously. Recombinant MCP-1, MIP-1a, and SDF-1a were obtained from Pepro Tech Ltd. (Rocky Hill, N.J.). Indo-1 AM was from Molecular Probes (Eugene, Oreg.). Hank's Balanced Salt Solution (HBSS) and RPMI 1640 were purchased from Gibco BRL (Grand Island, N.Y.). Ficoll Paque and Percoll solution were obtained from Amersham Pharmacia Biotech AB (Uppsala, Sweden). Unless otherwise stated, all other reagents were purchased from Sigma Chemical Co. (St. Louis, Mo.).

B. Preparation of Human Monocytes.

Human monocytes were purified from venous blood of normal volunteers essentially as described previously (Nakagawara, et al., J. Clin. Invest. 68:1243-53 (1981)). In brief, after erythrocytes were sedimented by addition of 6% dextran saline solution (I part to 5 parts heparinized blood), the leukocytes were collected, washed twice, and resuspended in Ca²⁺ and Mg²⁺-free HBSS containing 5% autologous serum. Mononuclear cells were acquired by centrifugation of the leukocyte suspension on Ficoll Paque at 1,500 rpm for 15 min. The cells were resuspended in RPMI 1640 containing 10% autologous serum and allowed to adhere to sterile tissue culture plates for 30 min in a humidified incubator at 5% CO₂ and 37° C. After incubation, non-adherent cells were removed by washing the plates three times with PBS at 37° C. Greater than 98% of the adherent cells showed the characteristic appearance of monocytes when examined by light microscopy following Wright staining or neutral red staining. To detach and harvest the adhered monocytes, 1 mM EDTA-PBS containing 5% serum was added and the plates were incubated on ice for 30 min. The monocytes were washed twice with HBSS and resuspended in RPMI 1640 with 0.1% autologous serum for the migration assay. The viability of monocytes was determined by trypan blue exclusion and was more than 98%. In some experiments, monocytes were purified according to another method using a Percoll discontinuous gradient described previously (Chuluyan & Issekutz, J. Clin. Invest. 92:2768-77 (1993)). No difference was noted in the purity and viability of the cells prepared by these two different methods.

C. Preparation of Human Cultured Peripheral Blood Macrophages and Alveolar Macrophages.

Human macrophages were obtained by culturing peripheral blood monocytes in vitro for 7 days as previously described (Fantuzzi, et al., Blood 94:875-83 (1999)). Human alveolar macrophages were obtained from bronchoalveolar lavage (BAL) fluid according to a previously described protocol (Sugimoto et al, Am. Rev. Respir. Dis. 139:1329-35 (1989)). The purity of the macrophages was over 90% and the viability was over 99%.

D. Migration Assay In Vitro.

Monocyte migration was examined by using 96-well micro Boyden chambers with 5 μm-pore size filters (Neuro Probe, Inc., Gaithersburg, Md.) as described previously (Falk, et al., J. Immunol. Meth. 33:239-47 (1980)), Chertov, et al., J. Biol. Chem. 271:2935-40 (1996)). Briefly, after the indicated concentrations of galectin-3 in RPMI 1640 were applied to the lower chambers, purified monocyte suspensions (2.5-5.0×10⁴/well) were applied to the upper chambers. After incubation of the chambers for 1 h in a humidified incubator at 5% CO₂ and 37° C., the filters were washed once with PBS and processed with Wright stain. The number of monocytes on the bottom side of the filters was counted in 5 to 10 high-power fields. Monocyte migration was calculated from the average numbers of the counted cells and expressed as % of input cells in a well.

In assays using inhibitory reagents, the purified monocytes were pretreated with or without the indicated concentrations of B2C10 (Liu, et al., Biochemistry 35:60773-79 (1996)) or anti-DNP IgG1 (Liu, et al., J. Immunol. 124:2728-31 (1980)) as an isotype-matched control mAb, galectin-3C, or PTX at 37° C. for 30 min. Then the cells were applied to the upper chambers in the presence of these inhibitors at the same concentrations used in the pretreatment. In the assays using lactose and sucrose, the sugars were added to the lower chambers at the initiation of the migration assay.

E. Migration Assay In Vivo.

The mouse air pouch experiments were performed according to a method described previously (Perretti, et al., J. Immunol. 151:4306-14 (1993)). Briefly, an air pouch was induced on the back of Balb/c mice by injecting 3 ml of air intradermally 2, 4, and 6 days before the experiments. Then, 1 ml of 0.9% sodium chloride (USP grade saline, Baxter Healthcare Corporation, Deerfield, Ill.) containing 1 μM galectin-3 was injected into the pouch. As positive and negative controls, 100 ng/ml of recombinant MCP-1 and diluent only, respectively, were injected. Four h afterwards, recruited cells were recovered by gently lavaging the pouch with 1 ml of PBS containing 1 mM EDTA. Cell number was determined and the distribution of leukocyte types was analyzed after cytospin preparation and Wright staining.

F. Measurement of Ca²⁺ Influx in Monocytes.

Intracellular concentrations of Ca²⁺ were measured by using Indo-1 AM according to a previously described method (Lopez, et al., Cytometry 10:165-73 (1989)). Purified monocytes were resuspended in HBSS containing 1 mM Ca²⁺, 1 mM Mg²⁺, and 5% autologous serum, and incubated with 10 mM Indo-1 AM for 45 min at 37° C. The cells were washed once, resuspended in the same buffer, and stimuli and inhibitors were added at the time points specified in the Figure Legends. Intracellular Ca²⁺ concentration was measured by monitoring light emission at 405 and 485 nm to an excitation wavelength of 355 nm, using an AMINCO-Bowman series 2 luminescence spectrometer (Rochester, N.Y.).

G. Data Analysis.

Data are summarized as the mean±Standard Deviation (SD). The statistical examination of the results was performed by the variance analysis using Fisher's protected least significant difference test for multiple comparisons. The analysis of the results from the mouse air pouch experiments was conducted with the Mann-Whitney test. p values of <0.05 were considered significant.

### Example 1

### Galectin-3 Induces Monocyte Migration In Vitro

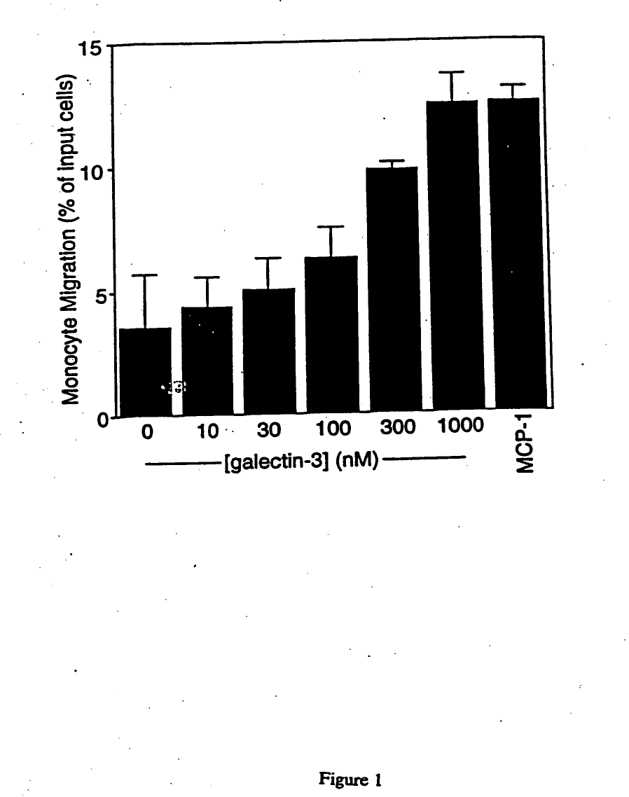

Using a micro Boyden chamber assay, human recombinant galectin-3 induced monocyte migration in a dose-dependent manner. Galectin-3 significantly increased monocyte migration at concentrations greater than 100 nM compared with diluent (control, 3.54±2.2% vs. 100 nM, 6.25±1.3%; 300 nM, 9.8±0.33%; 1 μM, 12.4±1.2%; p<0.05; n=4 experiments) (FIG. 1). While the difference in the effect between lower concentrations of galectin and control was not statistically significant in these initial experiments, in many subsequent ones, 10 nM galectin-3 also significantly increased monocyte migration (control, 4.26±1.3% vs. 10 nM, 7.01±2.1%; p<0.001; n=21). The effect of 1 μM galectin-3 on monocyte migration was comparable to that of human recombinant MCP-1, a strong chemoattractant for monocytes (Zachariae, et al., J. Exp. Med 171:2177-82 (1990)), at 100 ng/ml (11.6 nM) (FIG. 1), which was determined in dose-response experiments to be the concentration that induced maximum monocyte migration in this assay.

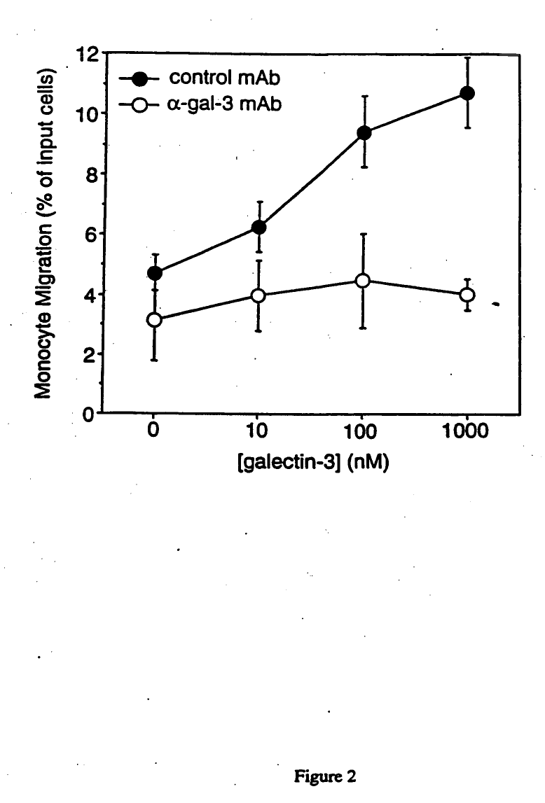

To rule out the possibility that the above results were due to contaminating bioactive substances such as heat-stable endotoxins in the recombinant galectin-3 preparations, experiments were conducted using galectin-3 samples pretreated at 100° C. for 5 min, which is known to inactivate this lectin (Yamaoka, et al., J. Immunol. 154:3479-87 (1995)). These samples did not induce monocyte migration at any of the concentrations used (10 nM-1 μM) (data not shown). Furthermore, the effect of an anti-galectin-3 mAb B2C10, which has been shown to block the binding of galectin-3 to IgE and neutrophil cell surfaces (Liu, et al., Biochemistry 35:60773-79 (1996)), on monocyte migration was studied. 10 μg/ml of B2C10, but not an isotype-matched control mAb, completely inhibited monocyte migration induced by galectin-3 at all concentrations examined (p<0.05, n=3) (FIG. 2). B2C10 did not affect MCP-1-induced monocyte migration significantly. These results indicate that exogenous galectin-3 induces migration of human monocytes in vitro.

### Example 2

### Galectin-3 is Chemotactic at High Concentrations and Chemokinetic at Low Concentrations for Monocytes

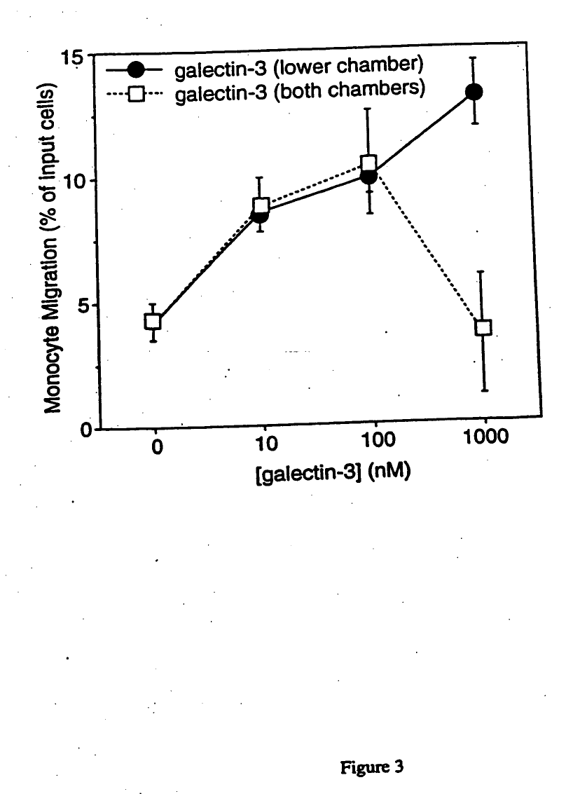

A checkerboard analysis was performed to assess whether galectin-3 is chemotactic or chemokinetic for monocytes. Various concentrations of galectin-3 were applied to the upper and/or lower chambers of a Boyden chamber, and monocyte migration was examined. As shown in Table 1 and FIG. 3, when 10 or 100 nM galectin-3 was used, no significant difference in monocyte migration was observed regardless of whether the protein was added to the lower chambers or to both chambers. In contrast, when 1 μM galectin-3 was added to both chambers, no significant increase in monocyte migration over the background was observed. These results indicate that the effect of galectin-3 in vitro is chemokinetic at low concentrations (10 and 100 nM), but chemotactic at high concentrations (1 μM).

TABLE 1
Checkerboard analysis of the effect of galectin-3 on the attraction
of human peripheral blood monocytes in vitro. Various concentrations
of galectin-3 were applied to the lower chambers and purified
monocytes mixed with various concentrations of galectin-3
were applied to the upper chambers, as described in Materials
and Methods. Monocyte migration is expressed as % migrated
cells of the total cells. Data are the mean ± SD of 4
individual experiments.

| | Above | | | |
|---|---|---|---|---|
| Below | 0 | 10 | 100 | 1000(nM) |
| 0 | 4.23 ± 0.75 | 7.55 ± 0.79 | 10.7 ± 0.86 | 3.30 ± 2.82 |
| 10 | 8.66 ± 0.22 | 8.88 ± 1.09 | 11.4 ± 2.11 | 3.25 ± 3.11 |
| 100 | 9.96 ± 0.72 | 9.23 ± 2.23 | 10.5 ± 2.10 | 4.55 ± 3.69 |
| 1000 | 13.1 ± 1.33 | 11.5 ± 3.49 | 12.5 ± 2.87 | 3.50 ± 2.41 |

### Example 3

### Necessity of N- and C-Terminal Domains of Galecin-3 for Monocyte Chemoattractant Activity

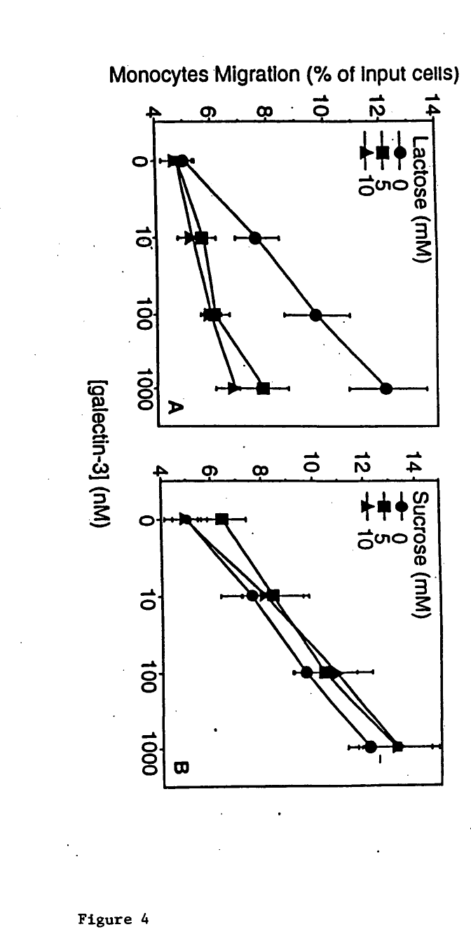

Galectin-3 is composed of a C-terminal lectin domain and an N-terminal non-lectin part. To determine whether the chemoattractant activity of galectin-3 is dependent on its lectin properties, the effect of saccharides on its induction of monocyte migration was tested. As shown in FIG. 4A, 5 mM lactose significantly decreased monocyte migration induced by 10 nM, 100 nM, and 1 μM galectin-3 by 63.8%, 71.5%, and 57.6%, respectively (p<0.05, n=3). Similarly, 10 mM lactose also significantly inhibited the migration by 78%, 74.1%, and 71.1%, respectively (p<0.05, n=3). These concentrations of lactose did not affect the monocyte migration induced by MCP-1. As a negative control, the effect of sucrose, which dose not bind to galectin-3, was also tested. As seen in FIG. 4B, sucrose had no significant effect on monocyte migration. These results indicate that the C-terminal lectin domain of galectin-3 is involved in the induction of monocyte migration.

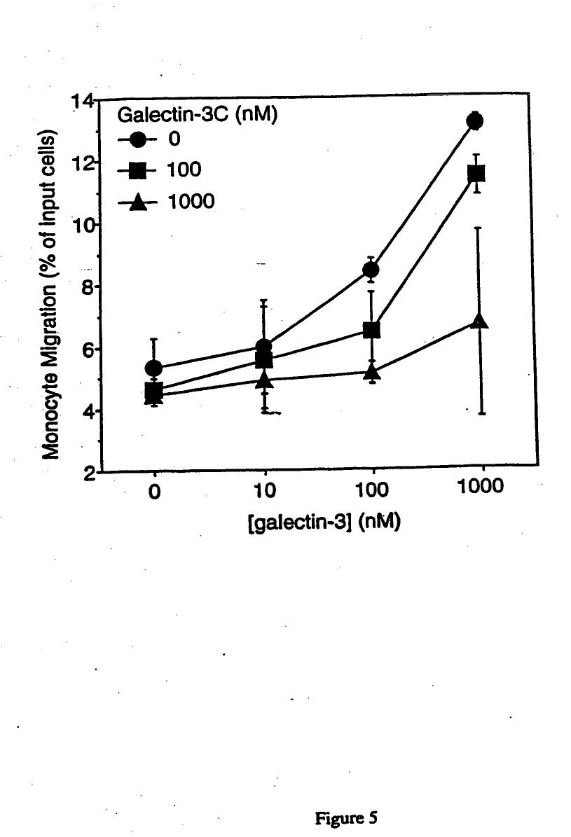

The effect of a recombinant C-terminal domain fragment of galectin-3 (galectin-3C) on monocyte migration was also examined. Monocytes were preincubated with various amounts of galectin-3C for 30 min at 37° C., the mixture was then applied to the upper chambers, and a standard migration assay was performed. As shown in FIG. 5, 1 μM galectin-3C alone did not have any chemokinetic effect on monocytes, but it significantly inhibited cell migration induced by 100 nM and 1 μM galectin-3 by 77.4% and 45.0%, respectively (p<0.05, n=3). Galectin-3C pretreated at 100° C. showed no effect on galectin-3-induced monocyte. No influence on monocyte migration was observed with 100 nM galectin-3C (FIG. 5). These results further confirm the involvement of the lectin domain in the chemoattractant activity and also suggest that the N-terminal domain is also necessary for this activity.

### Example 4

### Galectin-3 Induction of Monocyte Migration by PTX-Sensitive and -Insensitive Pathways

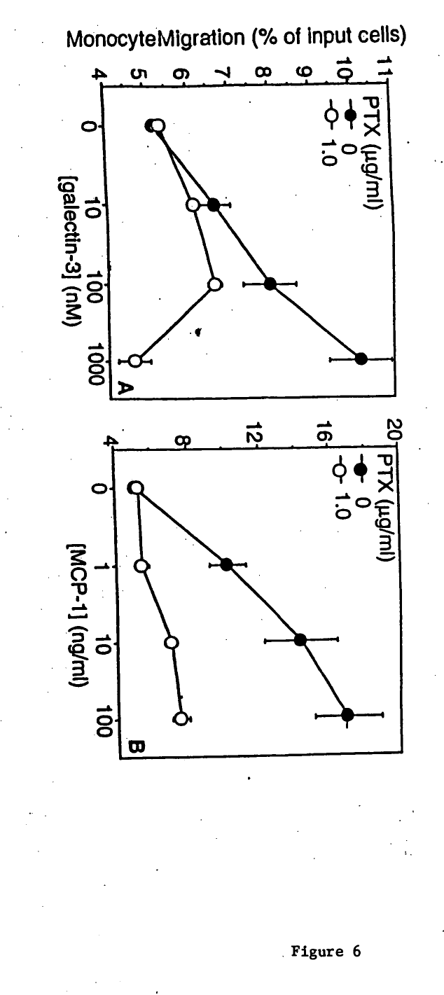

The possibility that G-proteins might be involved in galectin-3-induced monocyte migration was tested using the inhibitor pertussis toxin (PTX), because it is well known that many chemoattractants, including all chemokines, utilize G-protein-coupled receptors to transduce signals into the cell (Baggiolini, Nature 392:565-68 (1998)). Preliminarily, it was confirmed that 1 μg/ml of PTX did not decrease the viability of monocytes (data not shown). PTX decreased monocyte migration induced by 1 μM galectin-3 by 91.2% (p<0.01, n=5) (FIG. 6A). However, PTX did not significantly inhibit monocyte migration induced by 10 or 100 nM galectin-3 (p=0.8501 and 0.3093, respectively; n=5). In contrast, 1 μg/ml of PTX significantly inhibited monocyte migration induced by MCP-1 at all concentrations examined (FIG. 6B). These results indicate that a PTX-sensitive G-protein coupled receptor(s) is(are) involved in monocyte migration induced by high concentrations of galectin-3, but that a PTX-insensitive pathway(s) could be used in attracting monocytes by low concentrations of galectin-3.

### Example 5

### Galectin-3 Induced Increases in Intracellular Calcium Concentration by a PTX-Sensitive Pathway(s)

Galectin-3 can dimerize and crosslink cell surface receptors, suggesting that galectin-3 is chemotactic because it is able to activate chemokine receptors. To further analyze galectin-3-mediated signaling, the ability of this lectin to induce a Ca²⁺ influx in monocytes, because many chemoattractants are known to cause a Ca²⁺ influx. 1 μM galectin-3, but not lower concentrations, induced a Ca²⁺ influx in human monocytes similar to MCP-1 (FIGS. 7A, B), although the extent of the Ca²⁺ influx caused by the lectin was lower than that by the chemokine in all three separate experiments. Heat-inactivated galectin-3 did not produce any response (data not shown). The specificity of this activity was also demonstrated by the complete inhibition of galectin-3- but not MCP-1-induced Ca²⁺ influx by 5 mM lactose but not sucrose (FIGS. 7C, D). Furthermore, both the galectin-3- and MCP-1-induced Ca²⁺ influx was blocked by PTX (FIGS. 7E, F). These results indicate that galectin-3 causes a Ca²⁺ influx, which is probably mediated by a PTX-sensitive G-protein coupled receptor(s).

### Example 6

### Use of Known Chemokine Receptors on Monocytes by Galectin-3 to Induce Ca²⁺ Influx

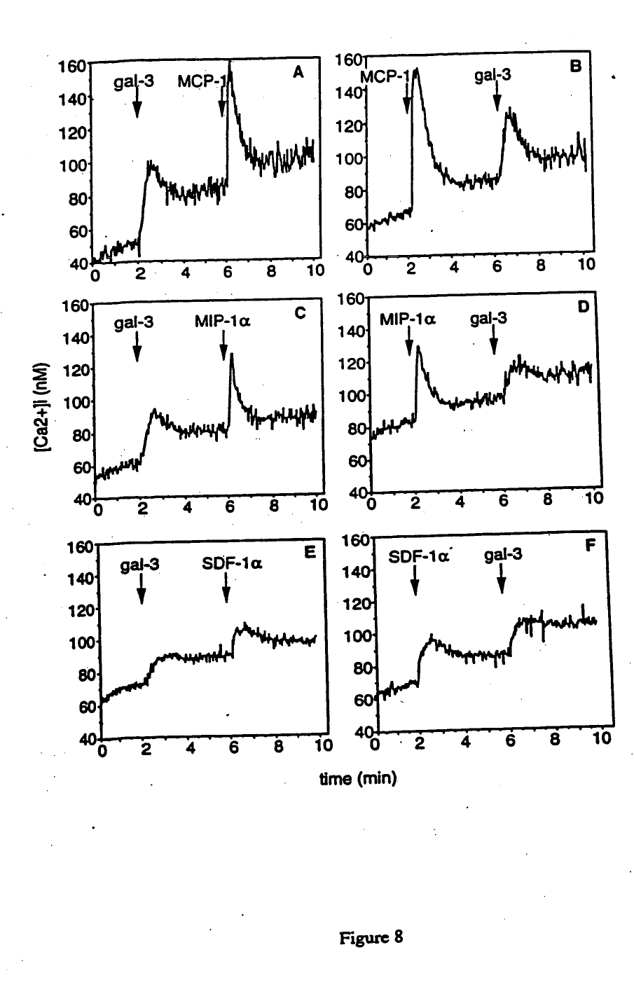

Among various chemoattractants, the monocyte/macrophage-reactive chemokines including MCP-1, MIP-1α, and SDF-1α are known to cause a Ca²⁺ influx in the cells (Sozzani, et al., J. Immunol. 150:1544-53 (1993); Bizzari, et al., Blood 86:2388-94 (1995); Oberlin, et al., Nature 382:833-35 (1996)) by binding to their receptors such as CCR2/9, CCR1/5/9, and CXCR-4, respectively, all of which are coupled with PTX-sensitive G-proteins (Baggiolini, Nature 392:565-68 (1998); Sallusto, et al., Immunol. Today 19:568-74 (1998); Zlotnik et al., Crit. Rev. Immunol. 19:147 (1999)). To determine the possibility that galectin-3 interacts with these receptors to transduce activation signal(s) into monocytes, Ca²⁺ influx experiments were performed to study cross-desensitization. This method is known to be useful in identifying the usage of the chemoattractant receptors, although cross-desensitization occurs at multiple levels and can affect signals mediated by other receptors (Richardson, et al., J. Biol. Chem. 270:27829-33 (1995); Tomhave, et al., J. Immunol. 153:3267-75 (1994)). All of the chemokines (100 ng/ml) induced a Ca²⁺ influx in human monocytes (FIGS. 8A, C, E). Responses were desensitized by the pretreatment with the same but not other chemokines, consistent with previous results from other investigators (Sozzani, et al., J. Immunol. 150:1544-53 (1993); Bizzari, et al., Blood 86:2388-94 (1995); Oberlin, et al., Nature 382:833-35 (1996)). However, there was no cross-desensitization between galectin-3 and any of the above-mentioned monocyte-reactive chemokines (FIG. 8A-F). These results suggest that galectin-3 does not interact with any of these presently known chemokine receptors expressed on monocytes for signal transmission into the cell.

### Example 7

### Induction of Macrophages Migration by Galectin-3, but not MCP-1

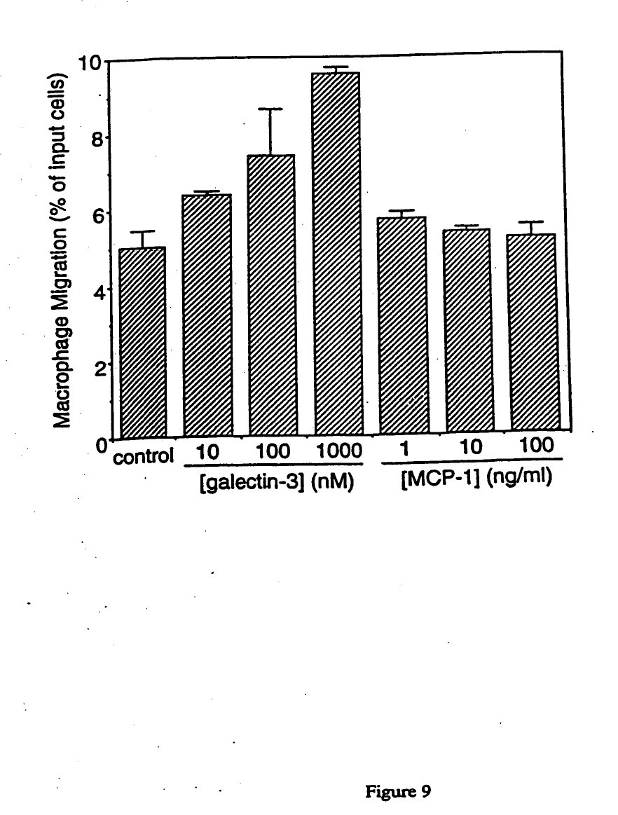

Unlike monocytes, few chemokines have been shown to attract mature macrophages (Zlotnik et al., Crit. Rev. Immunol. 19:147 (1999)). To determine the effect of galectin-3 on mature macrophages, human macrophages obtained from culturing peripheral blood monocytes as well as alveolar macrophages were used. Cultured human macrophages do not express a detectable amount of CCR2 and do not respond to its ligand MCP-1 (Fantuzzi, et al., Blood 94:875-83 (1999)), which we also confirmed (FIG. 9). In contrast, galectin-3 induced macrophage migration in a dose-dependent manner, and 1 μM galectin-3 enhanced the migration by 190% over that induced by the control medium (p<0.05, n=3) (FIG. 9). Similarly, human alveolar macrophages migrated towards galectin-3 in two separate experiments (FIG. 10). In these experiments, bell-shaped dose-response curves were obtained, which is commonly observed for many chemokines. In contrast, MCP-1 had no effect (FIG. 10, exp. 1) or a negligible effect (FIG. 10, exp. 2) on macrophage migration. These results indicate that galectin-3 but not MCP-1 is a chemoattractant for macrophages. The results also corroborate the conclusion made above that the signaling pathway induced by galectin-3 is not mediated through CCR2.

### Example 8

### Galectin-3 Induced Monocyte Migration In Vivo

The effect of galectin-3 on cell recruitment into mouse air pouches was examined to determine whether galectin-3 induces migration of cells in vivo. As shown in FIG. 11, galectin-3 increased the numbers of monocytes and neutrophils in the air pouch by 11.6 and 8.21 times, respectively, over those induced by vehicle (saline) only (p<0.05, n=4). In contrast, the numbers of lymphocytes and eosinophils were not augmented significantly by the treatment (p=0.309 and 0.112, respectively). These results indicate that galectin-3 selectively recruits monocytes and neutrophils in vivo.

### Example 9

### Galectin-3 Induced Macrophage Migration In Vivo

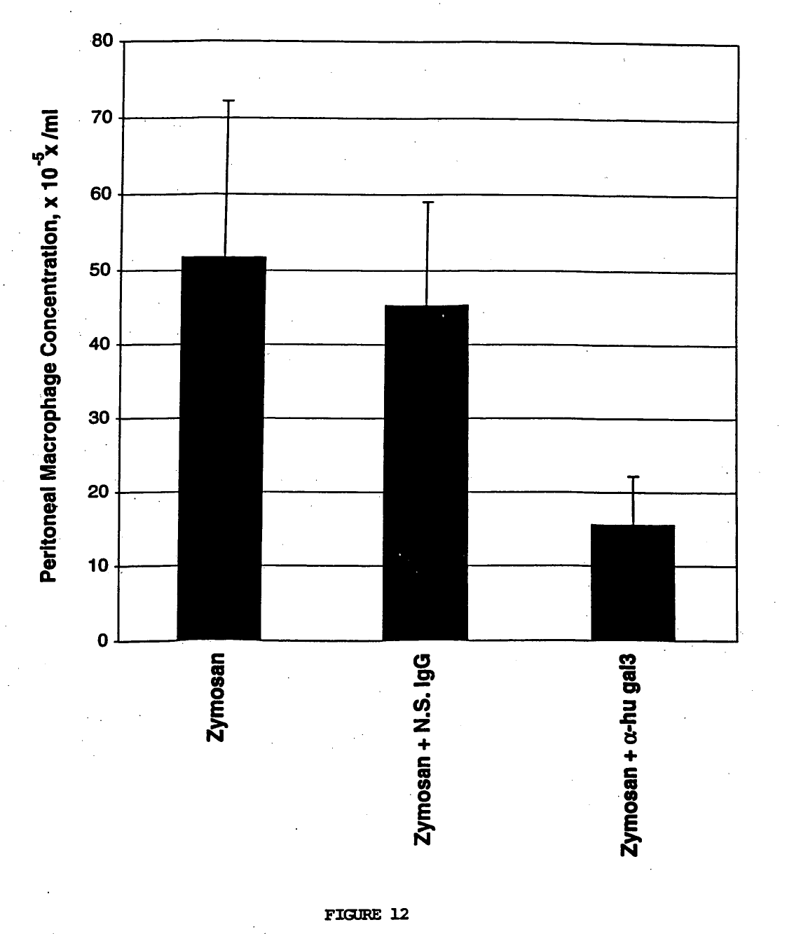

Briefly, mice were treated either with mouse monoclonal anti-galectin-3 antibody (B2C10) or isotype-matched nonspecific control antibody (300 μg/mouse) intraperitoneally. Thirty min after antibody treatment, zymosan (0.1 mg/g) was administered intraperitoneally. The following day, peritoneal lavage was performed with 3 ml of PBS and leukocytes contained in the recovered fluid were enumerated. As shown in FIG. 12, significantly fewer macrophages were recovered from the peritoneal cavity of mice treated with the anti-galectin-3 antibody (α-hu gal3) as compared to mice treated with control antibody (N.S. IgG). The results support a role for galectin-3 in regulation of macrophage infiltration during the inflammatory response, and are consistent with the previous finding that galectin-3 is a chemoattractant for monocytes/macrophages.

### Example 10

### Materials and Methods

This example describes various materials and methods.

Mice: Gal3^{-/-} mice were developed as described. (Hsu et al., Am J Pathol 156:1073 (2000)). These mice were backcrossed to C57BL/6 mice for nine generations and interbreeding of gal3^{+/-} F9 resulted in gal3^{+/+} and gal3^{-/-} mice in the C57BL/6 background, which were used throughout this study.

Immunization and Airway Antigen Challenge: The mice were immunized with 10 μg of OVA (grade V; Sigma, St. Louis, Mo.) in 2 mg of aluminum hydroxide gel intraperitoneally. The mice were placed in a Plexiglas chamber 10 to 14 days later, and subjected to aerosolized OVA (10 mg/ml) in saline administered by a nebulizer for 30 minutes each day for 3 to 6 days, as specified for the studies described in the figure legends. The control mice in all experiments received nonpyrogenic saline (Baxter, Deerfield, Ill.) at corresponding time points. In some studies, OVA from Sigma were compared with endotoxin-free OVA (ET-free OVA). This OVA was prepared by collecting chicken albumin aseptically and freeze-drying it in pyrogen-free vials. When macrophages were cultured with ET-free OVA, tumor necrosis factor-α secretion was not detectable, indicating absence of endotoxin.

For measurement of AHR, an intraperitoneal injection of 10 g of OVA mixed with 1 mg of aluminum hydroxide gel was administered on day 0 and an identical booster injection was given on day 7. Starting 7 days later, the mice were treated with aerosolized OVA (60 mg/ml) dissolved in phosphate-buffered saline (PBS, pH=7.4), or PBS, for 20 minutes per day in each of the subsequent 7 days. Control mice were treated with PBS alone. Treatment was initialized with an ultrasonic nebulizer (model 5000; DeVilbiss, Somerset, Pa.) into a plastic chamber that was 23×23×11 cm. The aerosol was delivered by providing ˜1 liter per minute (LPM) airflow at the nebulizer and excess aerosol escaped the box through a series of holes opposite the aerosol entry port.

Bronchoalveolar Lavage (BAL): BAL was performed 3 hours after the last airway antigen challenge. The BAL fluid obtained was centrifuged at 400×g to collect cells. The supernatant fluid was then centrifuged at 1000×g to remove cellular debris and stored at −70° C. until evaluated. Total viable cell numbers were determined by trypan blue exclusion. Differential cell counts were determined by staining cytospins with either Wright-Giemsa (Sigma) or Leukostat staining kit (Fisher Scientific Co., Pittsburgh, Pa.).

Histology: Lung tissue samples were fixed in 10% zinc-formalin (Biochemical Sciences, Inc., Swedesboro, N.J.) and paraffin-embedded. Goblet cells were stained and counted as previously described. (Jember et al., J Exp Med 193:387 (2001)). Briefly, 1 ml of 10% zinc-formalin (Fisher Scientific) was administered into the lungs via cannulated trachea. The small right lobe of the lung was dissected out, fixed in zinc-formalin, paraffin-embedded, and then sectioned, dewaxed, hydrated, stained with periodic acid-Schiff (PAS) stain, and counterstained with hematoxylin Gill no. 2 (Sigma). The goblet cells (both PAS+ and PAS−) around both the large and small bronchioles in each section were counted.

Immunohistochemistry was also performed with the paraffin-embedded sections. The endogenous peroxidase activity as well as nonspecific protein binding was sequentially blocked using 0.3% hydrogen peroxide and 5% normal goat serum, respectively. The sections were incubated with affinity-purified rabbit anti-galectin-3antibody (Frigeri et al., J Biol Chem 265:20763 (1990)) or normal rabbit IgG antibody (control) at 10 μg/ml for 30 minutes at room temperature and were then washed five times in PBS. Bound antibody was detected by sequential incubation with biotinylated goat anti-rabbit antibody and streptavidin-horseradish peroxidase followed by 3,3-diaminobenzidine (Biogenex Laboratories, San Ramon, Calif.). Slides were then washed in water and counterstained with hematoxylin Gill no. 2 (Sigma). For immunocytochemistry, cytospins of BAL fluid cells were stained according to a previously described method, (Liu et al., Am J Pathol 147:1016 (1995)) except that affinity-purified rabbit anti-galectin-3 antibody was used followed by steps as described above.

Quantitation of Galectin-3: Galectin-3 levels in BAL fluid were quantitated by enzyme-linked immunosorbentassay (ELISA) using a procedure similar to that described for human galectin-3. (Liu et al., Am J Pathol 147:1016 (1995)). Reagents used were affinity-purified goat anti-galectin-3 antibody as the capture antibody, affinity-purified rabbit anti-galectin-3 antibody (Frigeri et al., J Biol Chem 265:20763 (1990)) as the primary detection antibody, horse radish peroxidase-coupled goat anti-rabbit antibody (Zymed Laboratories, South San Francisco, Calif.) as the secondary detection antibody, and o-phenylene-diamine dihydrochloride as the substrate. Recombinant mouse galectin-3 was used as the standard.

Quantitation of Interleukin (IL)-4, Interferon (IFN)-γ, IgE, IgG₁, and IgG₂ₐ: IL-4 and IFN-γ levels in BAL fluid were measured by ELISA using commercial reagents (PharMingen, San Diego, Calif.) according to the manufacturer's protocol. Total IgE levels in BAL fluid and sera were determined by ELISA using affinity-purified goat and rabbit anti-IgE antibodies. (Liu et al., J Immunol 124:2728 (1980)). The OVA-specific IgG₁ and IgG₂ₐ antibodies in BAL fluids were detected on microtiter plates coated overnight with OVA at 10 μg/ml. The plates were blocked with 1% bovine serum albumin in PBS containing 0.05% Tween 20 for 2 hours at room temperature. Incubation of BAL fluid samples in OVA-coated wells was followed by biotin-labeled rabbit anti-mouse IgG₁ and IgG₂ₐ antibodies (Zymed Laboratories) each for 2 hours at room temperature. The plates were then incubated with horseradish peroxidase-avidin (Bio-Rad, Richmond, Calif.) followed by the horseradish peroxidase substrate o-phenylenediamine dihydrochloride (Sigma-Aldrich, St. Louis, Mo.) each for 30 minutes and read at 490 nm. The concentration of each Ig subclass in the samples was determined with the computer program SoftMaxPro provided with the plate reader (Molecular Devices, Sunnyvale, Calif.) and was read off a standard curve generated by incubating several concentrations of purified mouse Ig_{G1} or IgG₂ₐ in wells coated either with rat anti-mouse IgG₁ or rat anti-mouse IgG₂ₐ, respectively (CalTag Laboratories, Burlingame, Calif.) followed by biotinylated antibodies as above.

Measurement of Airway Responsiveness: Mice were anesthetized by an intraperitoneal injection of pentobarbital (180 mg/kg). After a surgical plane of anesthesia was achieved, the trachea was cannulated with a 19-gauge tubing adaptor attached to polyethylene tubing that passed through the plethysmograph chamber and was attached to a four-way connector, which was connected to a rodent ventilator (model 683; Harvard Apparatus, South Natick, Mass.) and pressure transducer. The ventilator was set to provide 150 breaths/minute with tidal volumes of 5 to 6 ml/kg and a positive end expiratory pressure of 3 to 4 cm H₂O. An internal jugular vein was cannulated with a saline-filled silicone catheter (0.021 cm OD, 6 to 8 cm in length, <0.005 ml volume) and attached to a 0.1-ml microsyringe. A 5×2-mm thoracotomy incision was made in a manner that allowed pleural pressure to equal body surface pressure. Flow was calculated by differentiation of the volume signal, transpulmonary pressure was measured as the difference of tracheal cannula and box pressure, and lung resistance was calculated as reported previously. (Martin et al., J Appl Physiol 64:2318 (1988)). Lung resistance (RL) was measured before and after each dose (26 to 34 μl volume) of intravenous methacholine (MCh). Percent baseline RL was calculated by dividing the greatest RL value obtained after MCh injection by the baseline value obtained immediately before and multiplying the result by 100.

Statistical Analysis: Statistical analysis of control and experimental groups was accomplished by Student's t-test using the software Statview 4.01 (SAS Institutes, Cary, N.C.). Changes in lung resistance to increasing concentrations of MCh were compared in mice using a two-factor repeated measures analysis of variance with the genetic strain and dose of MCh as the group factors. AP value less than 0.05 was considered significant.

### Example 11

### Galectin-3 Expression in the Airways is Up-Regulated During Allergic Airway Inflammation

This example describes Galectin-3 expression in the airways, which is up-regulated during allergic airway inflammation.

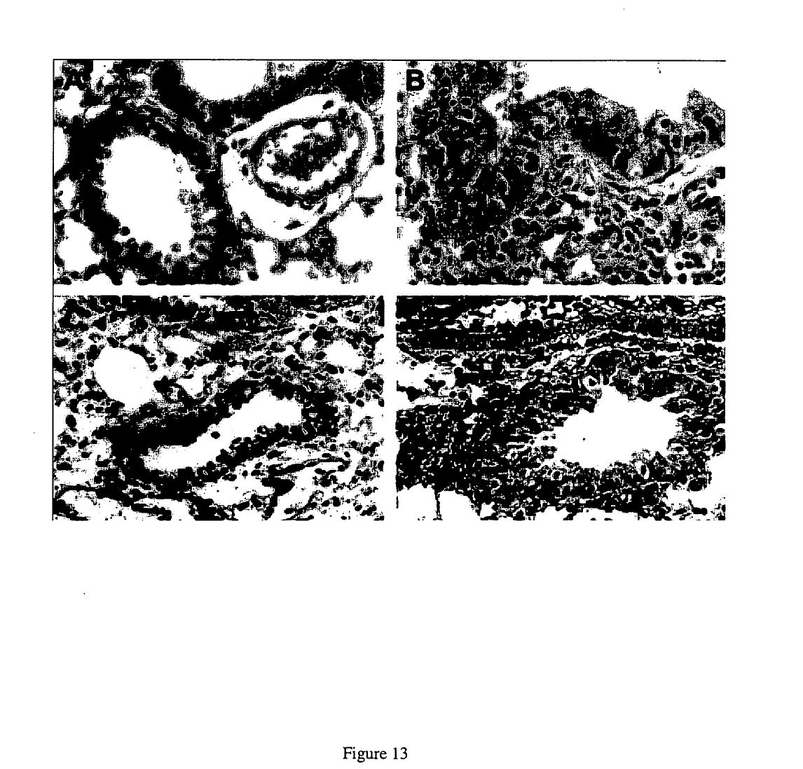

Lung tissue and BAL fluid from OVA-sensitized C57BL/6 mice challenged 14 days later with aerosolized OVA 30 minutes a day for 6 days. The control mice were treated with aerosolized saline. The mice were sacrificed 3 hours after the last antigen challenge. In contrast to the normal lungs from the control mice (FIG. 13A), the inflamed lungs (FIG. 13B) contained prominent peribronchial inflammatory cell infiltrations. Brown staining in C and D represents positive reactivity. Immunohistochemical analysis of galectin-3 expression showed that there was an increase in galectin-3 staining in the inflamed lungs (FIG. 13D) compared to the normal lungs (FIG. 13C). The increased staining is most likely because of infiltrating cells. No staining was observed when normal rabbit IgG was used instead of rabbit anti-galectin-3 antibody.

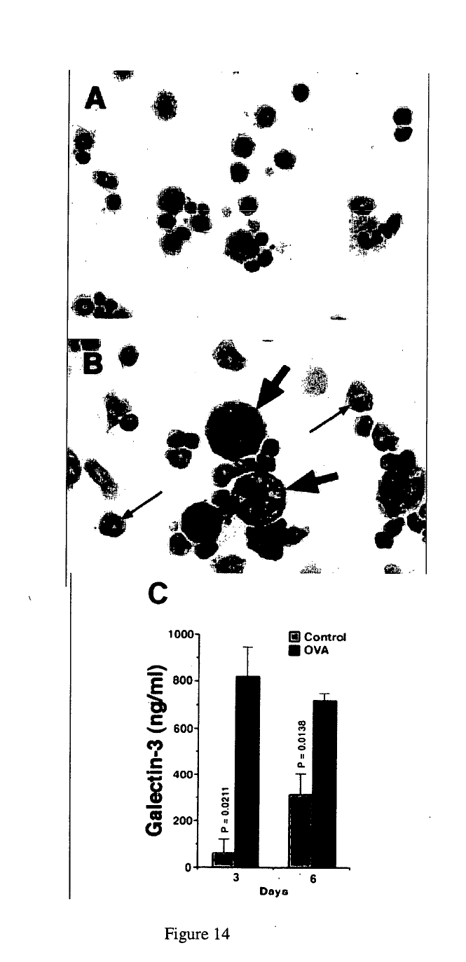

BAL fluid was obtained 3 hours after the last airway treatment in the studies described above. Macrophages are indicated by broad arrows and eosinophils are indicated by thin arrows. Brown staining in B represents positive reactivity. No staining was observed when normal rabbit IgG was used instead of rabbit anti-galectin-3 antibody. Inflammatory cells in BAL fluid from mice with inflamed airways were mostly eosinophils, but monocytes/macrophages and a few lymphocytes were also present (FIG. 14A). Immunocytochemical staining for galectin-3 showed that macrophages were strongly stained, where as eosinophils were not stained (FIG. 14B). Finally, galectin-3 levels in BAL fluid from mice challenged with aerosolized OVA were significantly higher than that from mice treated with aerosolized saline (FIG. 14C). The specificity of the anti-galectin-3 antibody used in these analyses was confirmed by the fact that lung tissues and lavaged cells from gal3^{-/-} mice were not stained at all by this antibody.

To determine whether galectin-3 release into the airway secretions was influenced by presence of endotoxinin OVA, mice were challenged either with saline (group 1), regular OVA (group2), or ET-free OVA (group 3). The results showed that mice from both groups 2 and 3 developed comparable levels of airway inflammation as indicated by the amount of cellular infiltration. In addition, galectin-3 levels in BAL fluids obtained from both groups were similar and higher than that from group 1.

The results indicate that galectin-3 release by airway cells was not because of low levels of endotoxinin OVA.

### Example 12

### Gal3^{-/-} Mice Exhibit Significant Reduction in Airway Inflammatory Responses

This examples shows that Gal3^{-/-} mice exhibit significant reduction in airway inflammatory responses.

Gal3^{-/-} mice were compared with gal3^{+/+} mice to determine whether galectin-3 contributes to the airway inflammatory response. It has been previously reported that gal3^{-/-} mice do not exhibit any overt defects and the total numbers of lymphocytes, ratios of CD4⁺/CD8⁺ cells, and numbers of CD3⁺ cells in various lymphoid organs are comparable between gal3^{-/-} with gal3^{+/+} mice. (Hsu et al., Am J Pathol 156:1073 (2000)).

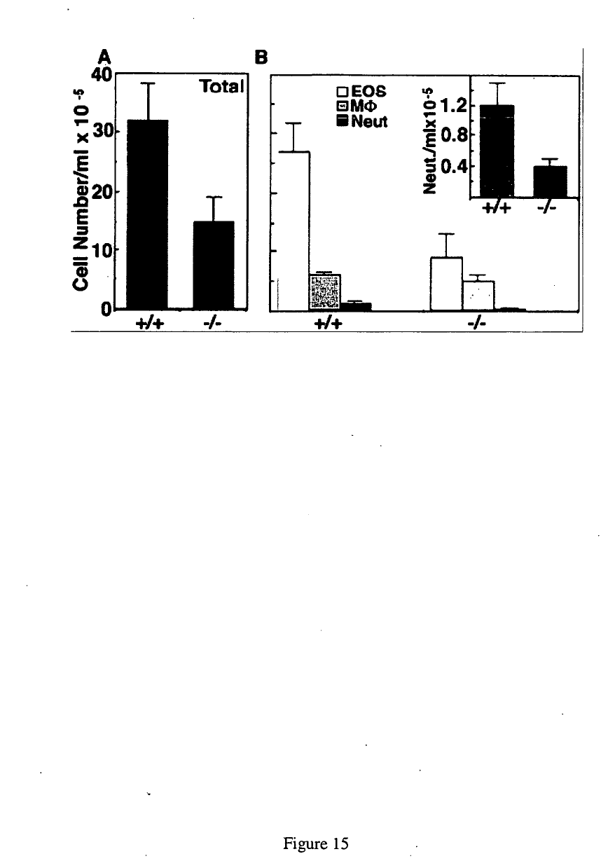

Mice were systemically immunized with OVA in aluminum hydroxide gel interperitoneally, and then challenged 14 days later with aerosolized OVA or saline 30 minutes a day for 3 days, and the inflammatory response was assessed by enumerating cells in BAL fluid. Both genotype controls challenged with aerosolized saline showed only a small number of cells in BAL fluid that were mostly monocytes. However, on challenging with aerosolized OVA, both genotypes mounted an inflammatory response, but gal3^{-/-} mice consistently showed significantly lower numbers of total inflammatory cells in BAL fluid compared to similarly challenged gal3^{+/+} mice (FIG. 15A). The difference was primarily because of eosinophils (FIG. 15B), but also partly because of neutrophils (FIG. 15B, inset), which represent only a small fraction of the leukocytes in BAL fluid. The numbers of monocytes/macrophages in BAL fluid were not significantly different between gal3^{-/-} with gal3^{+/+} mice (FIG. 15B).

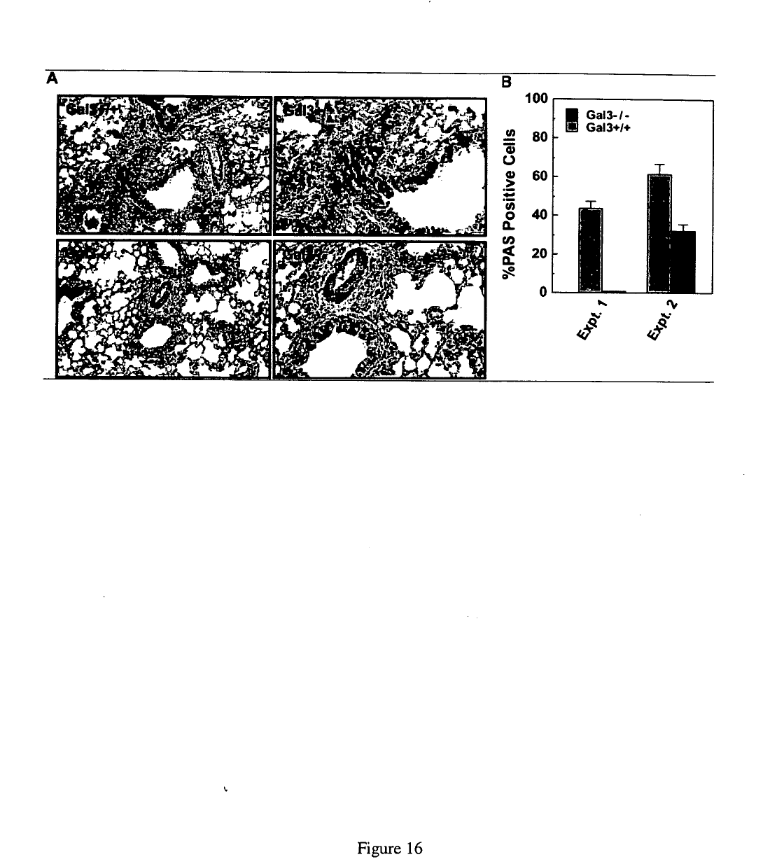

A characteristic feature of the murine model of asthma is goblet cell metaplasia with an accompanying increase in mucin production giving rise to mucous plugs in the airways. (Henderson et al., J Exp Med 184:1483 (1996)). OVA-sensitized mice were challenged 14 days later with aerosolized OVA given 30 minutes each day for 6 days. Three hours after the last aerosolized antigen challenge, the lung tissue were fixed and processed for PAS stain for mucin. Goblet cells of gal3^{+/+} mice stained more intensely than those from gal3^{-/-} mice, indicating higher mucin production per goblet cell in the former (FIG. 16A). In addition, the number of mucin-producing goblet cells in the lungs was significantly higher in gal3^{+/+} mice than gal3^{-/-} mice (FIG. 16B). The results suggest that gal3^{-/-} mice developed significantly less airway inflammation and hyperresponsiveness after airway challenge compared to gal3^{+/+} mice.

### Example 13

### Galectin-3-Deficient Mice are Defective in the Development of AHR

This example describes data indicating that galectin-3-deficient mice are defective in the development of AHR.

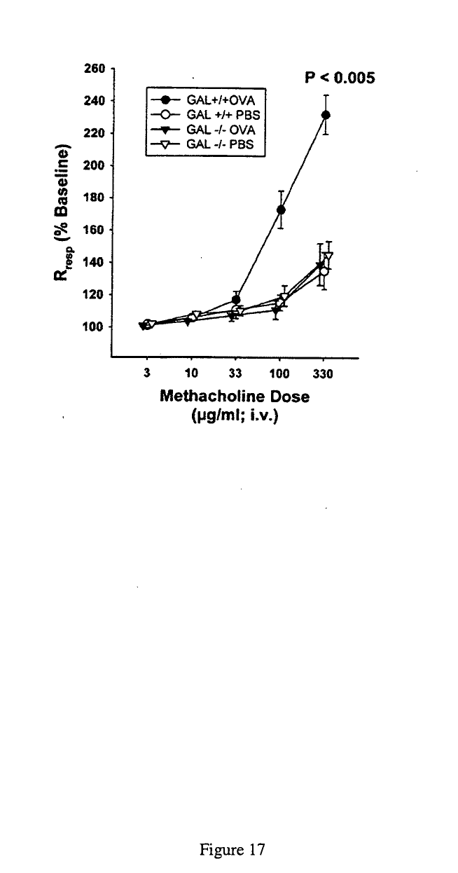

Development of AHR is another feature of human asthma consistently manifested in the murine model. (Willis Karp, Annu Rev Immunol 17:255 (1999)). Five gal3^{+/+} and eight gal3^{-/-} mice were immunized twice with OVA and then challenged with aerosolized OVA. Five mice for each genotype were exposed to aerosolized PBS instead of OVA. The airway response to MCh was measured by whole body plethys-mography. The OVA-sensitized mice were challenged with aerosolized OVA repeatedly and lung resistance (RL) was measured before and after each dose of intravenous MCh. Percent baseline RL was calculated by dividing the greatest RL value obtained after MCh injection by the baseline value obtained immediately before and multiplying the result by 100. P<0.005. Gal3^{-/-} mice developed a significantly lower degree of lung resistance in response to MCh challenge, compared to gal3^{+/+} mice (FIG. 17), suggesting that AHR to airway antigen challenge is ameliorated in mice with galectin-3 deficiency.

### Example 14

### Gal3^{-/-} Mice Develop a Lower Th2 Response but a Higher Th1 Response

This example describes data indicating that gal3^{-/-} mice develop a lower Th2 response but a higher Th1 response.

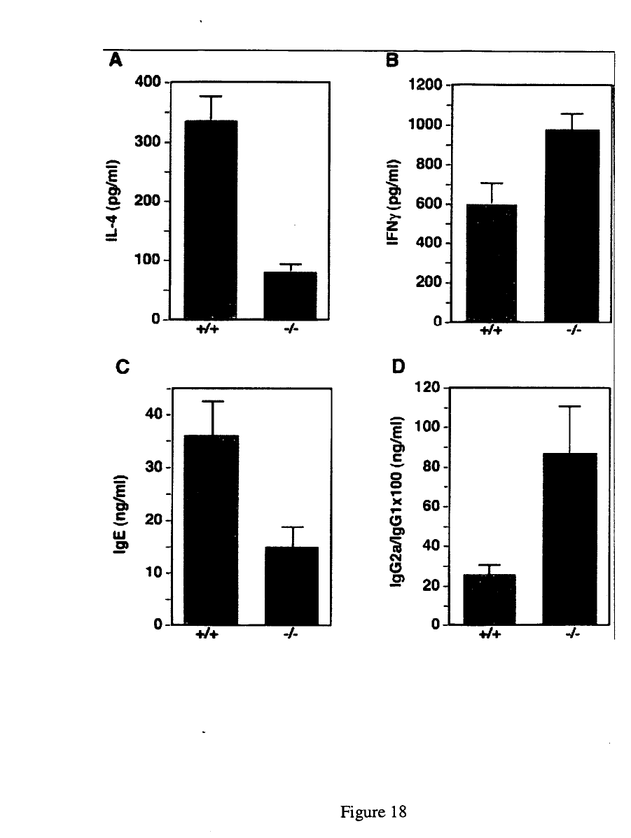

Th1 versus Th2 responses between gal3^{+/+} with gal3^{-/-} mice where compared to understand better the basis for the lower airway responses because of galectin-3 deficiency. First, the levels of cytokines in BAL fluid were examined. As shown in FIG. 18A, IL-4 levels in BAL fluid from gal3^{-/-} mice were significantly lower than those from gal3^{+/+} mice. In contrast, the opposite results were observed for IFN-γ (FIG. 18B).

It has been previously reported that BAL fluid from mice with allergic airway inflammation contained significant amounts of IgE, including antigen-specific IgE, which correlated well with the degree of airway inflammation. (Zuberi et al., J Immunol 164:2667 (2000)). Measurement of IgE levels in the BAL fluid thus represents a convenient and reliable way for assessing allergic airway inflammation. As shown in FIG. 18C, BAL fluid from OVA-challenged gal3^{-/-} mice contained significantly lower concentrations of IgE compared to identically treated gal3^{+/+} mice. The ratio of OVA-specific IgG₂ₐ (a Th1 antibody) to IgG₁ (a Th2 antibody) were measured and gal3^{-/-} mice were noted to have a higher ratio (FIG. 18D). In addition, cells from the lungs and the spleen from the OVA-challenged mice were obtained and cultured in the presence of OVA. The cells from gal3^{-/-} mice produced significantly higher amounts of IFN-γ (a Th1 cytokine) and lower amounts of IL-4 (a Th2 cytokine), compared to gal3^{+/+} mice. The results show that gal3^{-/-} mice have lower Th2 response, but higher Th1 responses compared to gal3^{+/+} mice, suggesting that galectin-3 regulates the Th1/Th2 response.

### Example 15

### Galectin-3-Deficient Mice that Exhibit a Lower IgE Response

This example describes galectin-3-deficient mice that exhibit a lower IgE response.

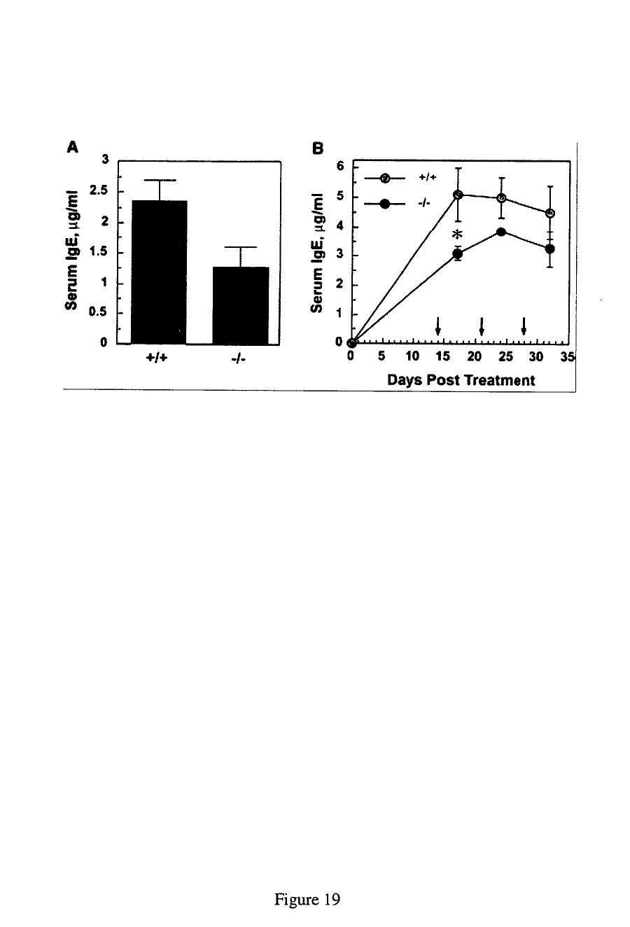

The IgE response in gal3^{+/+} and gal3^{-/-} mice were compared. Gal3^{-/-} mice sensitized with OVA and then challenged by aerosolized OVA exhibited lower serum IgE levels compared to similarly treated gal3^{+/+} mice (FIG. 19A). To determine whether the two genotypes differ in their IgE response to systemic immunization, mice were treated intraperitoneally with OVA in aluminum hydroxide gel and then challenged them intraperitoneally with the same antigen in aluminum hydroxide gel three times and evaluated the IgE levels in sera after the second through fourth immunizations. Gal3^{-/-} mice mounted a significantly lower IgE response after the secondary boost compared with gal3^{+/+} mice (FIG. 19B). The former continued to show suppressed IgE levels after each of the subsequent antigen challenges, although the differences became less pronounced at later time points.

1. A method for treating asthma, comprising administering to a subject having or at risk of having an acute or chronic asthmatic episode or an asthma associated symptom, an inhibitor of galectin-3 expression or activity in an amount sufficient to treat asthma.

2. The method of claim 1, wherein the inhibitor of galectin-3 activity comprises a galectin-3 subsequence that retains carbohydrate-binding activity.

3. The method of claim 1, wherein the inhibitor of galectin-3 activity comprises an N-terminal or C-terminal subsequence of galectin-3.

4. The method of claim 3, wherein the galectin-3 subsequence comprises a C-terminal portion of galectin-3.

5. The method of claim 1, wherein the inhibitor of galectin-3 activity comprises a peptide.

6. The method of claim 5, wherein the peptide is selected from:
SMEPALPDWWWKMFK; DKPTAFVSVYLKTAL; PQNSKIPGPTFLDPH;
APRPGPWLWSNADSV; GVTDSSTSNLDMPHW; PKMTLQRSNIRPSMP;
PQNSKIPGPTFLDPH; LYPLHTYTPLSLPLF;
LTGTCLQYQSRCGNTR; AYTKCSRQWRTCMTTH;
ANTPCGPYTHDCPVKR; NISRCTHPFMACGKQS; and
PRNICSRRDPTCWTTY.

7. The method of claims 2 or 3 or 5, wherein the galectin-3 subsequence or the peptide has from about 10-20, 20-30, 30-40, 40-50, 50-60, 60-75, 75-100, 100-150, 150-200 or more amino acid residues.

8. The method of claim 1, wherein the inhibitor of galectin-3 activity comprises galactose or a derivative thereof.

9. The method of claim 8, wherein the galactose derivative comprises a galactoside.

10. The method of claim 9, wherein the galactoside comprises a thio-galactoside or a thiodi-galactoside.

11. The method of claim 10, wherein the thio-galactoside is selected from:

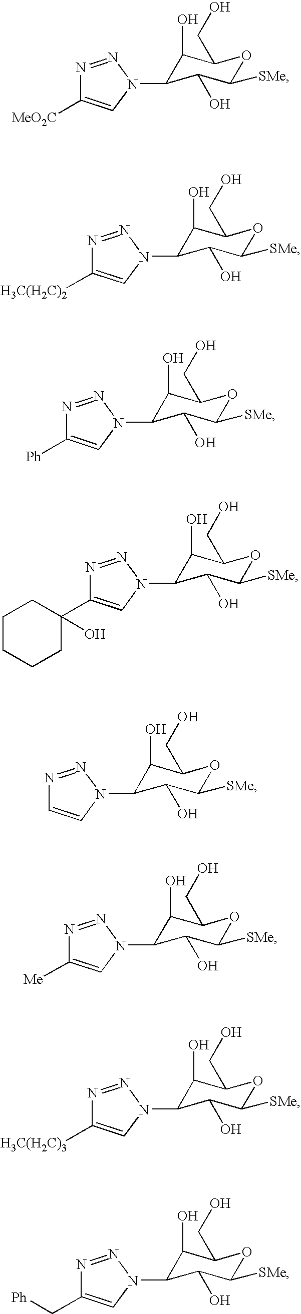

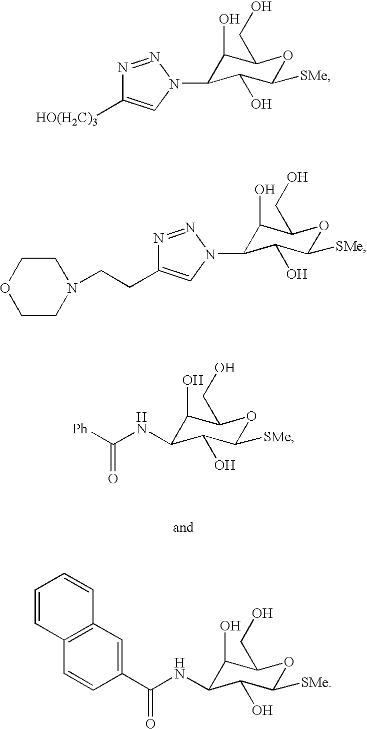

12. The method of claim 10, wherein the thiodi-galactoside is selected from:

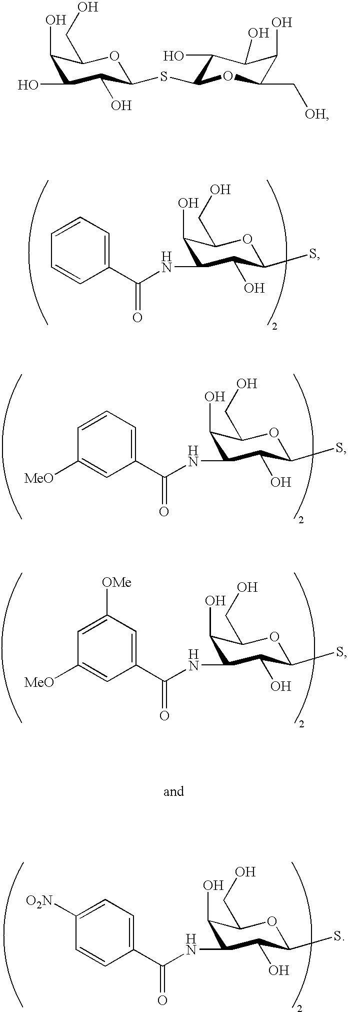

13. The method of claim 1, wherein the inhibitor of galectin-3 activity comprises a glycoconjugate, or derivative that binds galectin-3.

14. The method of claim 13, wherein the glycoconjugate comprises a glycolipid, a glycopeptide or a proteoglycan.

15. The method of claim 14, wherein the glycolipid is selected from any compound set forth in Table A.

16. The method of claim 14, wherein the glycopeptide is selected from any of compounds 1 to 33 of Table B.

17. The method of claim 1, wherein the inhibitor of galectin-3 activity comprises a monosaccharide, di-saccharide, tri-saccharide, polysaccharaide, or oligosaccharide.

18. The method of claim 17, wherein the saccharide comprises lactose, tetrasaccharide, beta-galactoside, or an analog or derivative thereof.

19. The method of claim 17, wherein the saccharide is naturally occurring or synthetic.

20. The method of claim 17, wherein the saccharide is selected from: Lactose; Galβ1,4GlcNAcβ1,3Galβ1,4Glc; Galβ1,3GlcNAcβ1,3Galβ1,4Glc; PNP βLacNAc; PNP βGalβ1,3GlcNAc; Galβ1,4GlcNAcβ1,3Gal; LacNAc; Galβ1,4GlcNAcβ1,2(Galβ1,4GlcNAcβ1,6)Man; MeβLacNAc; Galβ1,4GlcNAcβ1,2(Galβ1,4GlcNAcβ1,4)Manα1,3)(Galβ1,4GlcNAcβ1,2(Galβ1,4GlcNAcβ1,6)Manα1,6)Man; Galβ1,4Fru; Galβ1,4ManNAc; Galα1,6Gal; MeβGal; GlcNAcβ1,3Gal; GlcNAcβ1,4GlcNAc; Glcβ1,4Glc; and GlcNAc.

21. The method of claim 17, wherein the oligosaccharide is selected from any of compounds 1 to 33 of Table B.

22. The method of claim 1, wherein the inhibitor of galectin-3 comprises a glycodendrimer.

23. The method of claim 22, wherein the glycodendrimer is selected from:

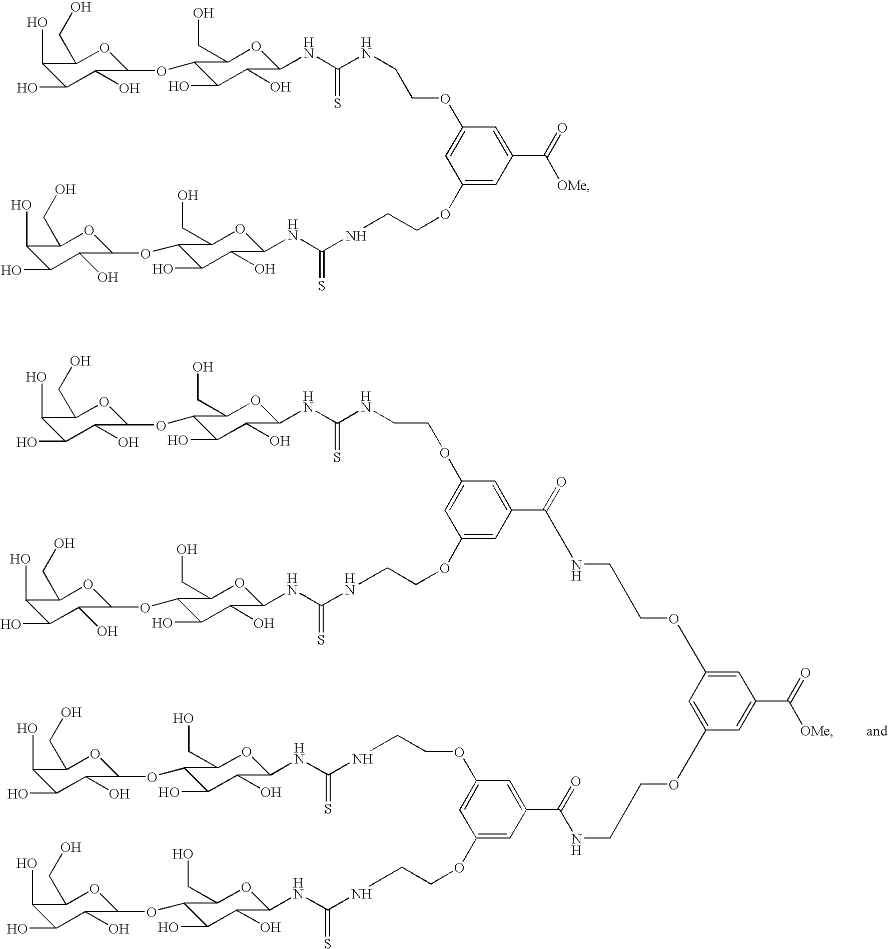

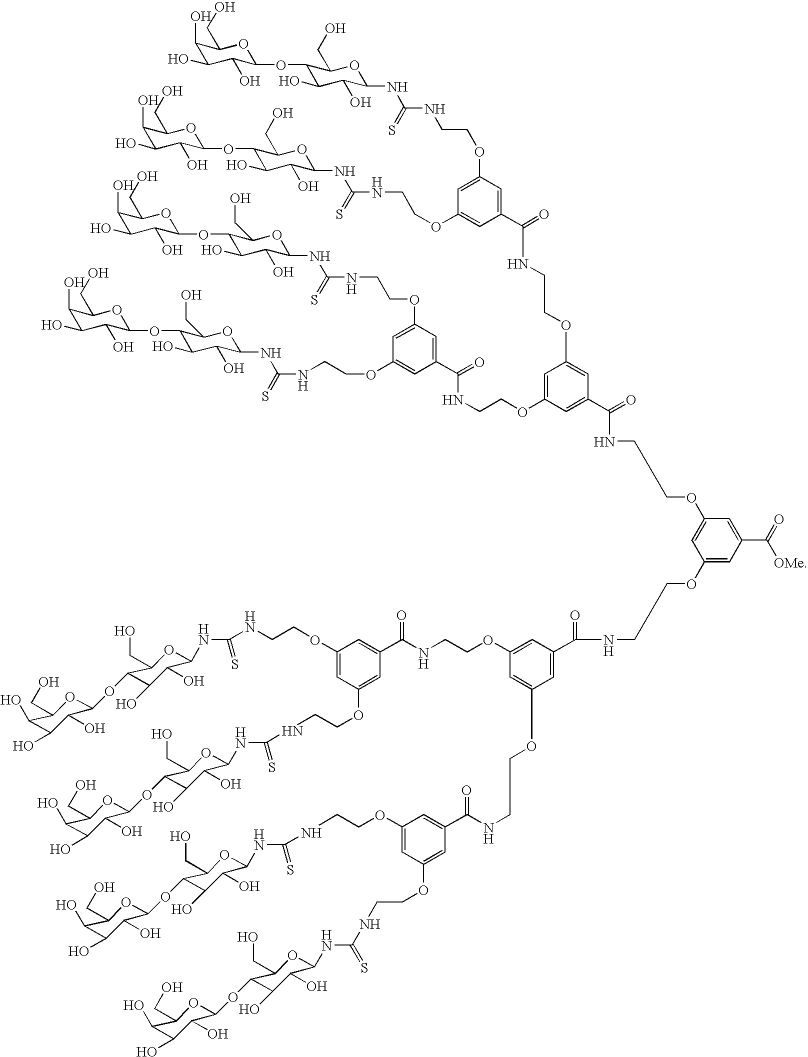

24. The method of claim 1, wherein the inhibitor of galectin-3 activity comprises N-acetyl lactosamine, or a derivative thereof.

25. The method of claim 24, wherein the N-acetyl lactosamine derivative comprises a C3′ amide, sulfonamide or urea derivative.

26. The method of claim 25, wherein the C3′ amide is selected from the group consisting of:

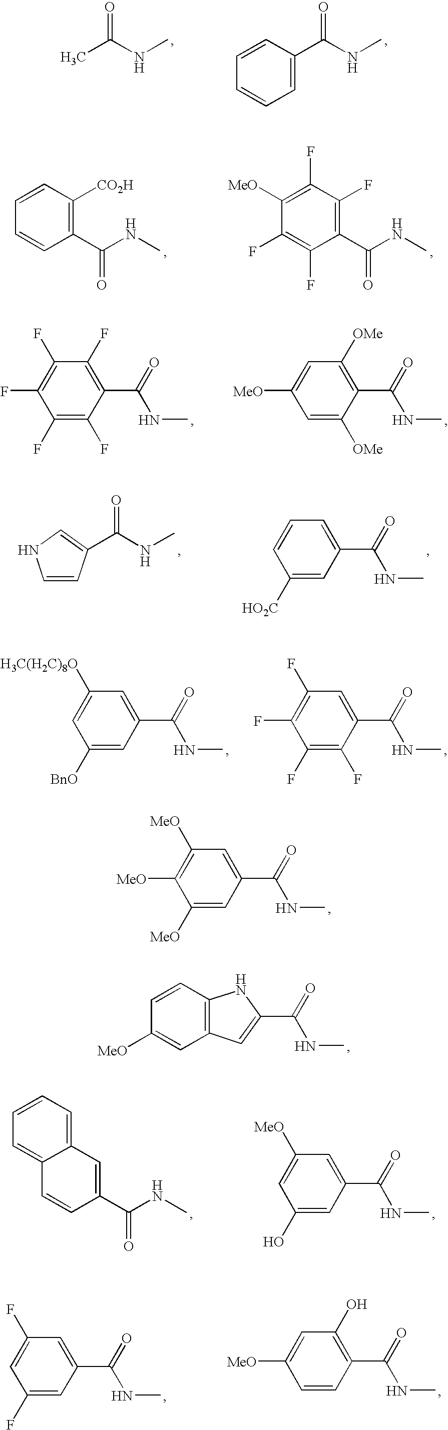

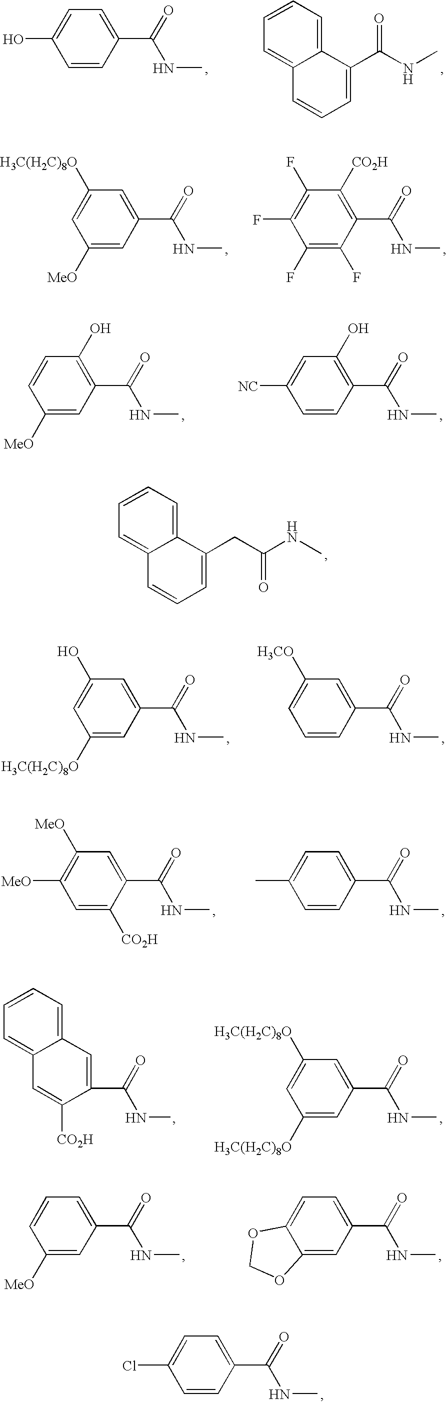

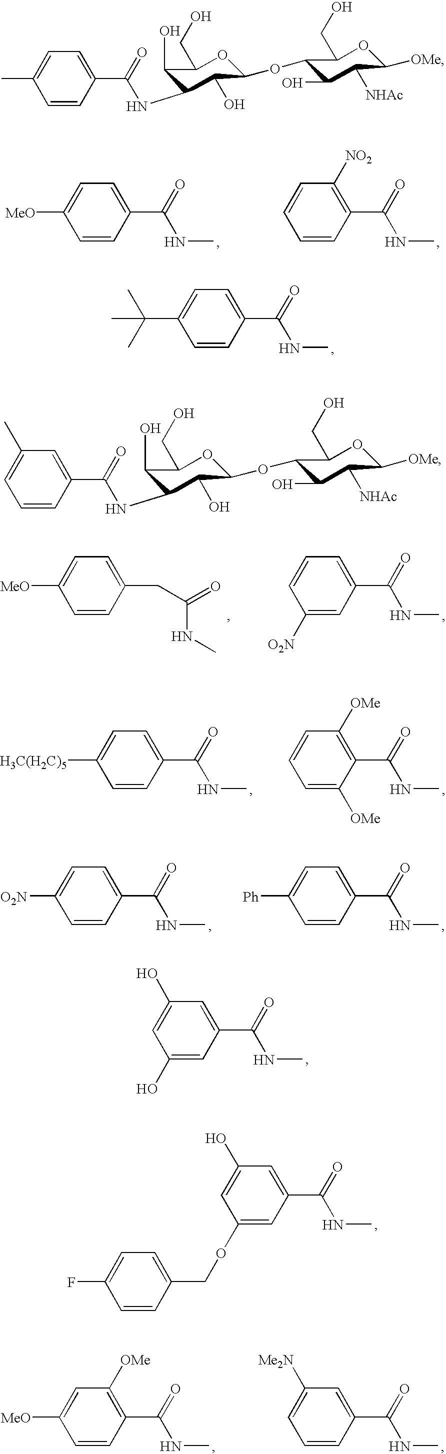

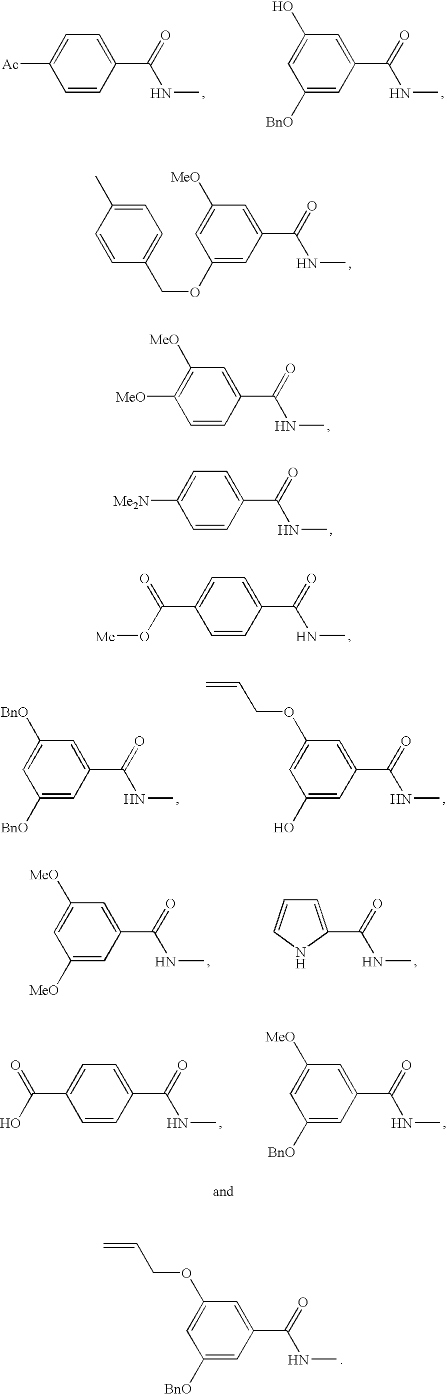

27. The method of claim 1, wherein the inhibitor of galectin-3 activity binds to galectin-3 at the carbohydrate-binding site.

28. The method of claim 1, wherein the inhibitor of galectin-3 expression or activity comprises a galectin-3 binding antisense nucleic acid, RNAi or triplex forming nucleic acid.

29. The method of claim 1, wherein the inhibitor of galectin-3 activity comprises an antibody or a fragment thereof that binds to galectin-3.

30. The method of claim 29, wherein the antibody that binds to galectin-3 is polyclonal or monoclonal.

31. The method of claim 29, wherein the antibody that binds to galectin-3 is selected from an IgG, IgA, IgM, IgE or IgD.

32. The method of claim 29, wherein the antibody fragment that binds to galectin-3 is selected from an Fab, Fab′, F(ab′)₂, Fv, Fd, single-chain Fvs (scFv), disulfide-linked Fvs (sdFv) and V_{L} or V_{H} sequence.

33. The method of claim 29, wherein the antibody is human, humanized or primatized.

34. The method of claim 29, wherein the antibody that binds to galectin-3 has the binding specificity of galectin-3 binding antibody B2C10.

35. The method of claim 29, wherein the antibody that binds to galectin-3 comprises an antibody that binds to an amino acid sequence to which B2C10 galectin-3 binding antibody binds.

36. The method of claim 29, wherein the antibody binds to galectin-3 N-terminal domain or C-terminal domain.

37. The method of claim 29, wherein the antibody that binds to galectin-3 inhibits galectin-3 oligomerization.

38. The method of claim 29, wherein the antibody that binds to galectin-3 inhibits galectin-3 binding to a carbohydrate.

39. The method of claim 1, wherein the inhibitor of galectin-3 further comprises a moeity that facilitates intracellular entry.

40. The method of claim 39, wherein the moeity that facilitates intracellular entry comprises a liposome or micelle, a poly-arginine sequence or an HIV tat sequence.

41. The method of claim 1, wherein the subject has previously experienced an asthmatic episode, allergic airway inflammation, airway- or broncho-constriction or obstruction, or is in need of airway- or broncho-dilation.

42. The method of claim 1, wherein the subject is experiencing an acute asthmatic episode, allergic airway inflammation, airway- or broncho-constriction or airway- or broncho-obstruction.

43. The method of claim 1, further comprising administering a drug to the subject.

44. The method of claim 1, wherein the inhibitor of galectin-3 expression or activity comprises a pharmaceutically acceptable carrier, excipient or diluent.

45. The method of claim 1, wherein the inhibitor of galectin-3 expression or activity comprises an article of manufacture.

46. A method of reducing or decreasing onset, progression, severity, frequency, duration or probability of one or more symptoms associated with asthma, comprising administering to a subject an amount of inhibitor of galectin-3 expression or activity sufficient to reduce or decrease onset, progression, severity, frequency, duration or probability of the one or more symptoms associated with asthma.

47-50. (canceled)

51. A method for treating a respiratory disorder or a respiratory airway or respiratory mucosal disorder, comprising administering to a subject having or at risk of having an acute or chronic a respiratory disorder or a respiratory airway or respiratory mucosal disorder or an associated symptom, an inhibitor of galectin-3 expression or activity in an amount sufficient to treat the respiratory disorder or the respiratory airway or respiratory mucosal disorder.

52-54. (canceled)

55. A method of reducing or decreasing the probability, severity, frequency, duration or preventing a subject from having an acute asthmatic episode, comprising administering to a subject that has previously experienced an asthmatic episode or has been diagnosed as having asthma with an amount of an inhibitor of galectin-3 expression or activity sufficient to reduce or decrease onset, probability, severity, frequency, duration or prevent an acute asthmatic episode.

56. (canceled)

57. A method of inducing or increasing airway-dilation, comprising administering to a subject in need of increased airway-dilation an amount of an inhibitor of galectin-3 expression or activity sufficient to induce or increase airway-dilation in the subject.

58. A method of reducing or decreasing probability, severity, frequency, duration or preventing airway-constriction or obstruction, comprising administering to a subject in need of reducing the probability, severity, frequency, duration or preventing airway-constriction or obstruction an amount of an inhibitor of galectin-3 expression or activity sufficient to reduce or decrease the probability, severity, frequency, duration or prevent airway-constriction or obstruction in the subject.

59-89. (canceled)
